(19) United States
(12) Patent Application Publication
(10) Pub. No.: US 2023/0310135 A1
(43) Pub. Date: Oct. 5, 2023

(54) THREE-DIMENSIONAL (3D) HERNIA PLUG DEVICE AND A METHOD OF MANUFACTURING THEREOF

(71) Applicants: 2350902 Alberta Ltd., Sherwood Park (CA); The Governors of the University of Alberta, Edmonton (CA)

(72) Inventors: Omar Farooq, Sherwood Park (CA); Cagri Ayranci, Edmonton (CA); Ahmed Qureshi, Edmonton (CA); Muhammad Bilal, Grande Prairie (CA); Lindsey Westover, Edmonton (CA)

(21) Appl. No.: 17/886,881

(22) Filed: Aug. 12, 2022

Related U.S. Application Data
(60) Provisional application No. 63/326,006, filed on Mar. 31, 2022.

(51) Int. Cl. A61F 2/00

(52) U.S. Cl. CPC A61F 2/0063; A61F 2230/0039; A61F 2210/0019; A61F 2230/005; A61F 2250/0039; A61F 2230/0069; A61F 2230/0093; A61F 2240/001; A61F 2220/0008

(57) ABSTRACT

Various embodiments are disclosed herein that generally relate to a three-dimensional (3D) hernia plug device and a method of manufacturing thereof. In at least one embodiment, there is disclosed a three-dimensional (3D) hernia plug, comprising: a waist portion extending, along a longitudinal extension axis, between a first waist end and a second waist end; one or more first overhangs coupled to the first waist end; and one or more second overhangs coupled to the second waist end, wherein the hernia plug is configured to translate between a pre-activation state and a post-activation state.

### FIELD

The described embodiments generally relate to hernia repair devices and, in particular, to a three-dimensional (3D) hernia plug device and a method of manufacturing thereof.

### BACKGROUND

The following is not an admission that anything discussed below is part of the prior art or part of the common general knowledge of a person skilled in the art.

Hernias are medical defects characterized by the protrusion of an internal organ, or other body part, through a muscle or tissue normally containing the organ or body part. In general, hernias are classified into various types based on their location. For example, the various types of hernias can include: (i) femoral hernias (e.g., hernias occurring at the top of the inner thighs); (ii) umbilical hernias (e.g., hernias occurring near the naval); (iii) hiatal hernia (e.g., hernias occurring at the chest, through the diaphragm); and (iv) inguinal hernias (e.g., hernias occurring in the groin region, and the area between the lower part of the abdomen and the thigh). Among these hernia classes, inguinal hernias are among the most common type and affects all genders.

To this end, when a hernia is detected, surgical procedures are often necessary to treat the hernia defect. While there is no definitive standard for these procedures, a typical procedure can involve securing conventional meshes to the hernia site, e.g., by means of sutures, tacks, or adhesives. These procedures, however, can be complex and can result in undesired complications to the patient.

### SUMMARY OF VARIOUS EMBODIMENTS

According to one broad aspect, there is disclosed a three-dimensional (3D) hernia plug, comprising: a waist portion extending, along a longitudinal extension axis, between a first waist end and a second waist end; one or more first overhangs coupled to the first waist end; and one or more second overhangs coupled to the second waist end, wherein the hernia plug is configured to translate between a pre-activation state and a post-activation state.

In at least some embodiments, the hernia plug is configured to change shape between the pre-activation state and a post-activation state.

In at least some embodiments, in the pre-activation state, each of the one or more and second first overhangs extends substantially parallel to the extension axis.

In at least some embodiments, in the post-activation state, each of the one or more first and second overhangs extends substantially orthogonal to the extension axis.

In at least some embodiments, the hernia plug translates from the pre-activation state to the post-activation state in response to a thermal stimuli.

In at least some embodiments, the thermal stimuli is an ambient temperature in a range of internal body heat.

In at least some embodiments, the thermal stimuli is an ambient temperature in a range of 35° C. to 40° C.

In at least some embodiments, the hernia plug is manufactured from a shape memory material.

In at least some embodiments, the thermal stimuli corresponds to the glass transition temperature (T_{g}) of the smart memory material.

In at least some embodiments, the smart material polymer comprises a shape memory polyurethane (SMPU).

In at least some embodiments, the smart material polymer comprises an MM4520 SMPU.

In at least some embodiments, in the pre-activation state, the hernia plug is configured to be receivable inside of a laparoscopic tube.

In at least some embodiments, the hernia plug is configured to be deployed at a hernia-defect site in the post-activated state.

In at least some embodiments, the hernia-defect site is an inguinal hernia-defect side, and the one or more first overhangs are configured to engage a groin-side of an abdominal wall, and the one or more second overhangs are configured to engage an abdomen-side of the abdominal wall, such that the hernia plug is self-gripping.

In at least some embodiments, each of the one or more first and second overhangs comprises one of a planar member and a rod-like elongate member.

In at least some embodiments, the waist portion comprises one of, (i) one or more connected continuous members, and (ii) one or more spaced discrete members.

In at least some embodiments, the waist portion comprises a continuous members having a circular cross-sectional profile in a plane orthogonal to the extension axis, and a hollow interior.

In at least some embodiments, the waist portion expands in an axis orthogonal to the extension axis in the post-activated state.

According to another broad aspect, there is provided a method of manufacturing a three-dimensional (3D) hernia plug, comprising: fabricating, using a shape memory material, the hernia plug in a post-activation shape, wherein the printing occurs in an ambient temperature that is below a glass transition temperature (T_{g}) of the smart polymer material; deforming the hernia plug into a pre-activation shape, wherein the deforming occurs while the ambient temperature is above the glass transition temperature (T_{g}); and reducing the ambient temperature is to below the glass transition temperature (T_{g}) to allow the pre-activation shape to correspond to a temporary shape of the hernia plug.

In at least some embodiments, the shape memory material comprises a shape-memory polyurethane (SMPU).

In at least some embodiments, the smart material polymer comprises an MM4520 SMPU.

In at least some embodiments, in the post-activation shape, the hernia plug is printed to comprise: a waist portion extending, along a longitudinal extension axis, between a first waist end and a second waist end; one or more first overhangs coupled to the first waist end; and one or more second overhangs coupled to the second waist end, and each of the one or more first and second overhangs extends substantially orthogonal to the extension axis.

In at least some embodiments, in the pre-activation state, each of the one or more and second first overhangs extends substantially parallel to the extension axis.

In at least some embodiments, the fabrication further comprises fabricating the hernia plug using a secondary support material.

In at least some embodiments, after the fabrication and before the deforming, the method further comprises removing the secondary support material.

In at least some embodiments, the secondary support material comprises water-soluble material, and removing the secondary support material comprises exposing the fabricated hernia plug to water.

In at least some embodiments, the fabrication is performed using a three-dimensional (3D) printer.

In at least some embodiments, the 3D printer is a 3D Fused Deposition Modeling (FDM) printer.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A is a schematic illustration of an example inguinal hernia;

FIG. 1B is a schematic illustration of an example conventional two-dimensional (2D) mesh applied to a hernia defect site;

FIG. 1C is a schematic illustration of an example three-dimensional (3D) mesh applied to a hernia defect site;

FIG. 1D is an example plot showing the change in elastic module of an example thermo-responsive smart polymer material (SMP) in response to temperature change;

FIG. 1E is a simplified block diagram illustrating the process for pre-programming an SMP;

FIG. 2A is a simplified illustration of a two-dimensional (2D) projection of an example embodiment of a 3D hernia plug, and showing the 3D hernia plug in a post-activated state, in accordance with at least some embodiments described herein;

FIG. 2B is a simplified illustrations of the hernia plug of FIG. 2A in a pre-activated state;

FIG. 2C is a simplified illustrations of the hernia plug of FIG. 2A between a pre-activated and post-activated state, in accordance with at least some embodiments;

FIG. 3 is a schematic illustration of a hernia plug, in a pre-activated state, and received inside of a conveyance device, in accordance with some teachings provided herein;

FIGS. 4A-4F are various schematic illustrations of an example process for deploying a 3D hernia plug at a hernia defect site, in accordance with the teachings provided herein;

FIG. 5A is a perspective view of a 3D hernia plug, in accordance with an example embodiment;

FIG. 5B is an elevation view of the 3D hernia plug of FIG. 5A;

FIG. 5C is a perspective cross-sectional view of the 3D hernia plug of FIG. 5A, taken along the section line 5-5′ in FIG. 5A;

FIG. 5D is a top-down view of the 3D hernia plug of FIG. 5A;

FIG. 6A is a perspective view of a 3D hernia plug, in accordance with another example embodiment;

FIG. 6B is an elevation view of the 3D hernia plug of FIG. 6A;

FIG. 6C is a perspective cross-sectional view of the 3D hernia plug of FIG. 6A, taken along the section line 6-6′ in FIG. 6A;

FIG. 6D is a bottom-up perspective view of the 3D hernia plug of FIG. 6A;

FIG. 6E is a top-down view of the 3D hernia plug of FIG. 6A;

FIG. 6F is a bottom-down perspective view of the 3D hernia plug of FIG. 6A;

FIG. 7A is a perspective view of a 3D hernia plug, in accordance with another example embodiment;

FIG. 7B is an elevation view of the 3D hernia plug of FIG. 7A;

FIG. 7C is a perspective cross-sectional view of the 3D hernia plug of FIG. 7A, taken along the section line 7-7′ in FIG. 7A;

FIG. 7D is a bottom-up perspective view of the 3D hernia plug of FIG. 7A;

FIG. 7E is a top-down view of the 3D hernia plug of FIG. 7A;

FIG. 7F is a bottom-down perspective view of the 3D hernia plug of FIG. 7A;

FIG. 8A is a perspective view of a 3D hernia plug, in accordance with another example embodiment;

FIG. 8B is a bottom-up perspective view of the 3D hernia plug of FIG. 8A;

FIG. 8C is a top-down perspective view of the 3D hernia plug of FIG. 8A;

FIG. 8D is an elevation view of the 3D hernia plug of FIG. 8A;

FIG. 8E is a perspective cross-sectional view of the 3D hernia plug of FIG. 8A, taken along the section line 8-8′ in FIG. 8A;

FIG. 8F is a bottom view of the 3D hernia plug of FIG. 8A;

FIG. 8G is a top view of the 3D hernia plug of FIG. 8A;

FIG. 9A is a perspective view of a 3D hernia plug, in accordance with some other example embodiments;

FIG. 9B is a side elevation view of the hernia plug of FIG. 9A;

FIG. 9C is a perspective cross-sectional view of the plug in FIG. 9A, taken along the section line 9C-9C′ in FIG. 9A;

FIG. 9D is a top-down plan view of the hernia plug of FIG. 9A;

FIG. 9E is a bottom-up perspective view of the hernia plug of FIG. 9A;

FIG. 10 is a process flow for an example embodiment of a method for manufacturing a 3D hernia plug, in accordance with at least some embodiments;

FIG. 11 is a perspective view of an example 3D hernia plug with secondary support material;

FIG. 12 is a plot showing measured force versus displacement for various example 3D hernia plug samples;

FIG. 13 is a plot of a model calculation for a single compression test for an example 3D hernia plug sample;

FIGS. 14A-14C are various plots showing measurement of plastic deformation under cyclic loading for three different hernia plug samples; and

FIG. 15A-15D are various plots showing measurement of activations for short overhangs, long overhangs, short versus long overhangs and waist portions for different example 3D hernia plug samples.

Further aspects and features of the example embodiments described herein will appear from the following description taken together with the accompanying drawings.

### DETAILED DESCRIPTION OF THE EMBODIMENTS

Numerous embodiments are described in this application, and are presented for illustrative purposes only. The described embodiments are not intended to be limiting in any sense. The invention is widely applicable to numerous embodiments, as is readily apparent from the disclosure herein. Those skilled in the art will recognize that the present invention may be practiced with modification and alteration without departing from the teachings disclosed herein. Although particular features of the present invention may be described with reference to one or more particular embodiments or figures, it should be understood that such features are not limited to usage in the one or more particular embodiments or figures with reference to which they are described.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising” and variations thereof mean “including but not limited to,” unless expressly specified otherwise. A listing of items does not imply that any or all of the items are mutually exclusive, unless expressly specified otherwise. The terms “a,” “an” and “the” mean “one or more,” unless expressly specified otherwise.

As used herein and in the claims, two or more parts are said to be “coupled”, “connected”, “attached”, “joined”, “affixed”, or “fastened” where the parts are joined or operate together either directly or indirectly (e.g., through one or more intermediate parts), so long as a link occurs. As used herein and in the claims, two or more parts are said to be “directly coupled”, “directly connected”, “directly attached”, “directly joined”, “directly affixed”, or “directly fastened” where the parts are connected in physical contact with each other. As used herein, two or more parts are said to be “rigidly coupled”, “rigidly connected”, “rigidly attached”, “rigidly joined”, “rigidly affixed”, or “rigidly fastened” where the parts are coupled so as to move as one while maintaining a constant orientation relative to each other. None of the terms “coupled”, “connected”, “attached”, “joined”, “affixed”, and “fastened” distinguish the manner in which two or more parts are joined together.

Further, although method steps may be described (in the disclosure and/or in the claims) in a sequential order, such methods may be configured to work in alternate orders. In other words, any sequence or order of steps that may be described does not necessarily indicate a requirement that the steps be performed in that order. The steps of methods described herein may be performed in any order that is practical. Further, some steps may be performed simultaneously.

As used herein and in the claims, a group of elements are said to ‘collectively’ perform an act where that act is performed by any one of the elements in the group, or performed cooperatively by two or more (or all) elements in the group.

As used herein and in the claims, a first element is said to be “received” in a second element where at least a portion of the first element is received in the second element unless specifically stated otherwise.

Some elements herein may be identified by a part number, which is composed of a base number followed by an alphabetical or subscript-numerical suffix (e.g. 112ₐ, or 112₁). Multiple elements herein may be identified by part numbers that share a base number in common and that differ by their suffixes (e.g. 112₁, 112₂, and 112₃). All elements with a common base number may be referred to collectively or generically using the base number without a suffix (e.g. 112).

Hernias are defects caused by a weakening or breach of a tissue or muscle wall. As explained in the background, the various types of hernias include, by way of example, femoral hernias, umbilical hernias, hiatal hernias and inguinal hernias.

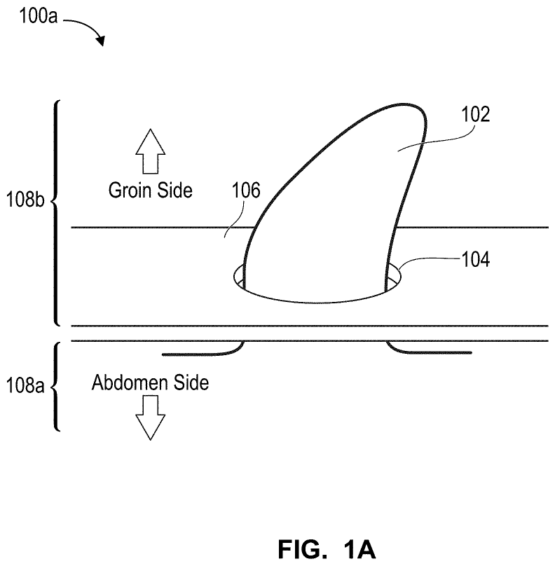

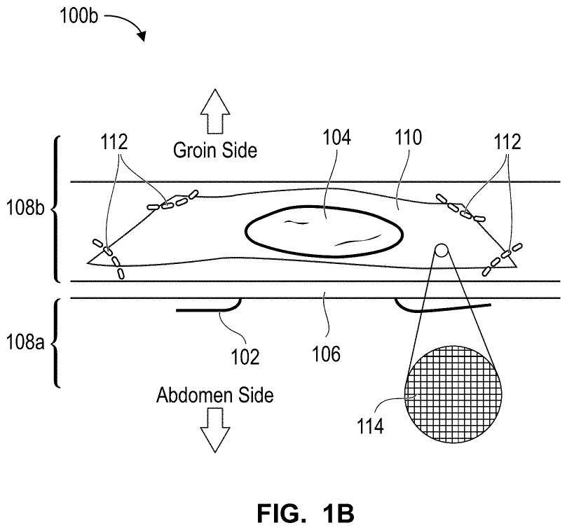

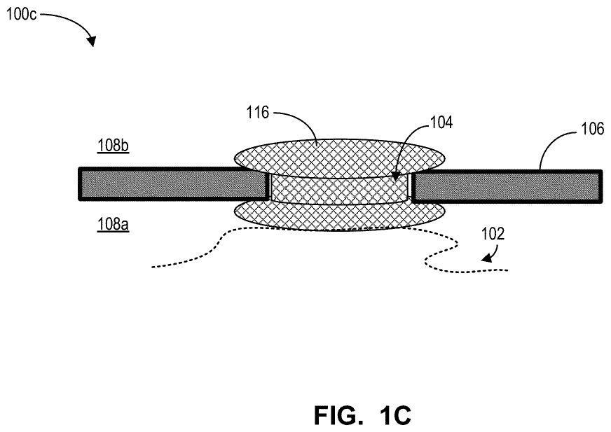

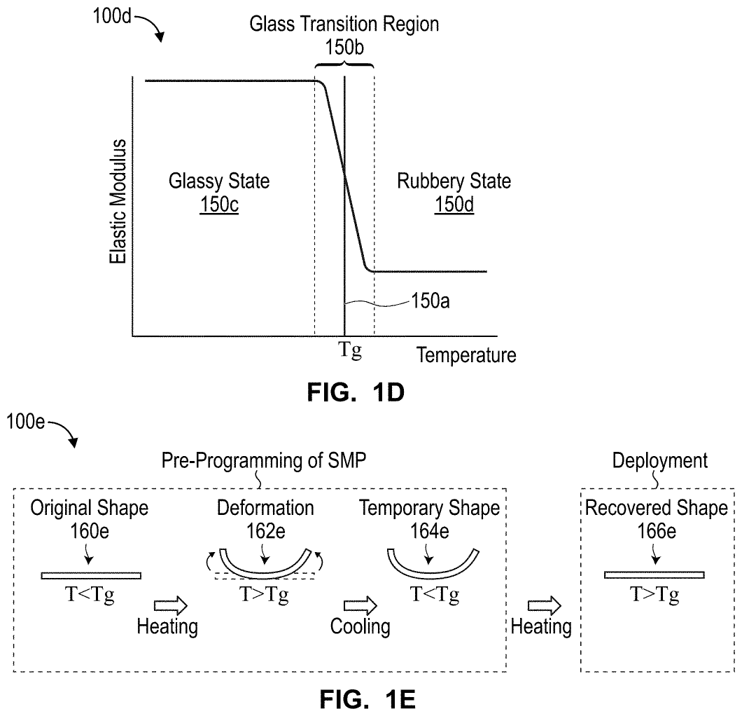

In the specific case of inguinal hernias, the breached or weakened tissue wall is the lower abdominal wall. FIG. 1A shows a schematic illustration 100a of an example inguinal hernia. As shown, an internal organ 102 protrudes through the breach 104 in the abdominal wall 106, such that the organ 102 protrudes from the lower abdomen side 108a, and into the groin side 108b.

In general, attempts at treating hernias have focused on developing tools which minimize patient pain, reduce complications rates and simplify procedure complexity. While there is no universally accepted “gold standard” in the hernia repair industry, over the years, several techniques have made their way as common good practices to characterize the treatment of this disease.

One example common practice is the use of “tension repair”. In tension repair, an abdominal incision is made over the hernia site, and the protruding tissue 102 is pushed back into its normal location 108a. The surgeon then directly stitches the edges of the defect together, or tissue wall 106, using multiple sutures as required, e.g., the surgeon sews the defect shut. While this technique appears to be straightforward, it has been discarded in most developed countries due to its high recurrence rate and the post-operative pain experienced by patients.

To mitigate the drawbacks of tension repair, hernia defects are now often treated using “tension-free repair”. In tension-free repair, a prosthetic mesh is applied at the location of the hernia site 104. Thereafter, the surgeon sutures around the circumference of the defect to secure the mesh, but without otherwise sewing the defect shut. By way of illustrative example, FIG. 1B shows a schematic illustration 100b of a two-dimensional (2D) mesh 110 applied over the breach area 104. The mesh 110 is composed of a plurality of small pores 114. Mesh 110 may be secured in place by one or more fixing mechanisms 112 (e.g., sutures) applied by the surgeon. As shown, the mesh 110 pushes the protruded organ 102 back into the abdomen side 108a so as to allow repair of the hernia defect.

In view of the foregoing, the primary advantage of tension-free repair is that it avoids the application of pressure on the muscular tissue surrounding the defect (e.g., by avoiding stitching together the tissue wall, as performed in tension repair), which could potentially weaken the region further.

Commercially available prosthetic meshes, used for tension-free repairs, are generally classified as being one of two types: (i) static meshes, or (ii) dynamic meshes. Static and dynamic meshes are distinguished based on their ability to move with the abdominal wall 106 and to accommodate the motility of the abdomen. More particularly, static meshes are typically fixed to the abdominal wall by means of sutures, tacks or adhesives, and do not accommodate the movement of the abdominal wall (e.g., FIG. 1B). In contrast, dynamic meshes may also be fixed to the abdominal wall, however, their structure has sufficient tolerance to allow for the natural motion of the abdominal wall and to resist tears to higher degree.

While the use of synthetic meshes (e.g., static or dynamic) has become the popular technique for hernia repair surgeries—there are still a number of important drawbacks associated with these meshes. For example, these drawbacks include high rate of recurrence, as well as post-operative pain owing to medical complications. The high recurrent rate and pain is often due to a combination of the shape of the 2D mesh, the use of securing mechanisms (e.g., sutures, etc.), as well as the complexity of the surgery required to install the meshes. In particular, the paper-like two-dimensional shape of the 2D mesh often prevents the mesh shape from flexibly deforming while the body moves, twists and/or bends. In turn, the mesh is likely to rip or damage with body motion, thereby causing recurrent patient pain.

To this end, the use of conventional meshes often results in poor quality of tissue regrowth, and is often associated with tearing and bleeding at the hernia site. In many cases, the typical biological response is a foreign body reaction, mainly caused by the fixation mechanisms used to hold the mesh in place, which in turn is correlated to high complication rates. Further, the treatment of inguinal hernias using static synthetic meshes, is also not entirely tension free, and is associated with tearing, bleeding, hematoma as well as nerve entrapment. These side-effects can cause severe discomfort and pain, instead of regenerating the weakened tissue.

In view of the foregoing, and to alleviate at least some of the problems associated with conventional meshes—recent innovations have focused on enhancing the following desired properties of meshes:
Material Properties: The selection of materials strongly influences the inflammatory response of the body, which in turn, determines recovery rates as well as pain experienced by patients.
(ii) Structural Properties: Structural properties are further subcategorized into (a) pore characteristics, and (b) the weight of the mesh. In respect of pore characteristics, the characteristics of the mesh pores (e.g., pores 114 in FIG. 1B) plays a critical role in influencing the limited dynamic movement of the mesh, which is caused by the collapse of the pores under strain. Further, mesh porosity is also a determining factor for the risk of infection. In respect of the weight of the mesh, the weight of synthetic meshes can be used for classifying meshes as either lightweight or heavyweight depending upon the polymer weight, and amount of raw material used. Lightweight meshes have a weight per unit area ranging from 35 to 70 g/m², standard weight meshes from 70 to 140 g/m² and heavyweight meshes being greater than 140 g/m². Since lightweight meshes contain less material than their counterparts, they induce a moderate foreign body reaction in the body with decreased inflammatory response providing better tissue incorporation, reduced patient discomfort and increased prosthesis acceptance, providing a notable advantage over heavyweight meshes. While the mesh weight does not influence the rate of reoccurrence, patients with lightweight meshes have reported lower discomfort and an earlier return to activity as compared to heavyweight meshes.
(iii) Mechanical Properties: Some of the major mechanical properties which are considered while designing meshes are (a) tensile strength, (b) burst strength and (c) stiffness. While tensile strength is conventionally defined as the maximum force that can be applied before failure per unit area, for the purpose of meshes, it is usually reported in relations to clamping width with units usually being N/cm or equivalent. The burst strength is defined as the maximum uniformly distributed pressure applied at a right angle to the surface of a material to withstand standardized conditions. Stiffness is defined as the ability of a material or object to resist deformation and can be calculated as the slope of the force versus displacement curve. Meshes with higher stiffness tend to dehisce from the abdominal wall when the patient moves

While there has been significant work on the mechanical, material and structural properties to improve desired properties of synthetic meshes, synthetic meshes still face inherent challenges that have not been addressed by these improvements. For example, foreign body reactions are still caused by the use of sutures, tacks or adhesive to secure the mesh in the hernia site and contribute to the complication rate in hernia repair procedures.

The development of three-dimensional (3D) meshes has been recently considered as a reasonable alternative to two-dimensional (2D) meshes. It is believed that 3D meshes may alleviate some of the drawbacks associated with 2D meshes.

FIG. 1C shows a schematic illustration 100c of an example three-dimensional (3D) mesh 116 applied in the hernia region 104.

As shown, the 3D mesh 116 protrudes through the hernia region 104 to push the organ 102 back inside the abdominal region 108a. The main advantage of the 3D mesh 116 is that it displays superior compliance to dynamic motion that is often absent from static meshes. In turn, the 3D mesh design can accommodate for increased motility, e.g., of the abdominal wall 106. Some 3D meshes have also shown to display a progressive development of artery and vein infrastructure in the hernia site with time. The growth of a broad network of arteries and veins is considered to be critical for healthy tissue regeneration, which requires significant vascular development. In many cases, 3D meshes have also been reported to reduce post-operative pain, and to minimize complication rates as well demonstrating absence of long-term chronic pain and patient discomfort.

Accordingly, with the potential advantages offered by 3D meshes, as compared to 2D meshes, a new wave of innovation has now been directed at an effort to further reduce the complications of hernia repair surgery using 3D meshes.

Current 3D meshes still, nevertheless, suffer from a number of important drawbacks. For example, many 3D mesh designs still require excessive operating time and procedure complexity to embed the mesh into the hernia defect site. Further, many 3D meshes may still require external securing mechanisms (e.g., suture or tacks) to secure the mesh, which contributes to patient discomfort and various complications (e.g., poor tissue recovery, inflammation, bleeding and further medical complications).

In view of the above, embodiments herein provide for a three-dimensional (3D) smart hernia plug. The disclosed 3D smart hernia plug is believed to mitigate at least some of the aforementioned drawbacks associated with conventional hernia treating devices.

In accordance with the teachings provided herein, the disclosed 3D hernia plug is manufactured from a smart material (thereby termed a “3D smart hernia plug”). More generally, the inventors have appreciated a novel opportunity for applying smart materials to the manufacture of hernia plugs. To the inventors' knowledge, the integration of SMPs has remained largely absent in the development of 3D mesh plugs.

In more detail, smart materials refer to a class of materials that can change physical properties—such as shape, color and size—in response to external stimuli. The external stimuli can comprise, for example, heat, electricity, stress, light, moisture, etc.

Shape-memory polymers (SMPs) are a special branch of smart materials, and display shape-memory effect (SME). SME is the ability of such a material to recover from a temporarily assigned shape, to an original shape, in response to the particular stimuli. SMPs that activate (e.g., change shape) in response to thermal stimuli are the most common type of SMPs, and are also known as “thermo-responsive” SMPs.

Reference is now briefly made to FIG. 1D, which better clarifies the mode of operation of thermo-responsive SMPs.

As noted above, in response to a temperature change, thermo-responsive SMPs may vary their shape between: (i) a temporarily assigned shape, and (ii) an original assigned shape. The transition temperature, which varies the shape of the SMP, is also known as the glass transition temperature (T_{g}).

FIG. 1D illustrates a plot 100d of the varying elastic modulus in a thermo-responsive SMP, and around the glass transition region 150b, e.g., defined around the glass transition temperature (T_{g}) 150a. As shown, when the temperature is below the glass transition region 150b (region 150c), the SMP is in a glassy or “stiff” state (also known as the “temporary state”). Alternatively, when the temperature is greater than the glass transition region 150b (region 150d), the SMP transitions to a rubbery or flexible state (also known as the “shape recovered state”). SMPs can be pre-designed to have a pre-programmed shape which is automatically adopted when the when the temperature is greater than the glass transition region 150b, e.g., the shape recovered state.

In accordance with at least some embodiments provided herein, the disclosed 3D hernia plug is manufactured from a thermo-responsive SMP. The SMP-based plug can change its shape between a temporarily-assigned shape (or a pre-activation state), and an originally-assigned shape (or a post-activation state), responsive to a thermal stimulus.

In particular, in the temporary, pre-activated state (150c in FIG. 1D), the disclosed plug has a generally compressed size. This, in turn, facilitates ease of delivery of the plug to the hernia affected region. Upon delivery, the body's internal temperature acts as a thermal stimulus, which automatically transforms the plug into its originally-assigned, or post-activated shape (150d in FIG. 1D). In this post-activated state, the plug can expand such as to effectively seal the hernia region. In this manner, the 3D hernia plug is configured with automatic, self-deploying capabilities.

To this end, the ability of the plug to self-deploy not only reduces surgical time, but also reduces surgical complexity (e.g., in transporting and deploying the plug within the hernia region). Further, the use of SMPs yields faster deployment. In at least one embodiment, the plug can achieve 90% deployment within four minutes of activation. The use of SMPs also allows the plug to be light weight, process with ease, and sustain higher recovery strain. As noted above, these factors have been appreciated as being important for effective repair of hernia defects.

In at least one embodiment described herein, in the post-activated state, the 3D hernia plug is configured to be “self-gripping”. That is, the hernia plug may self-lock into position to avoid displacement within the hernia region. In turn, the hernia plug does not require external securing mechanisms (e.g., sutures or tack) to retain its position within the hernia-affected area, e.g., is as required for conventional meshes. Accordingly, the proposed 3D hernia plug overcomes concerns related to patient discomfort due to the use of external securing mechanisms, as well as associated post-surgical complications (e.g., poor tissue recovery, inflammation, bleeding and further medical complications).

As also provided in greater detail herein, the disclosed 3D hernia plug may also be compatible with the more popular, and less invasive laparoscope assisted hernioplasty. For example, the proposed hernia plug is designed to fit, in the pre-activated state, in a tube similar to that of a laparoscope. Once inserted and pushed into the body, the hernia plug can automatically expand into the post-activated state due to thermal activation, thereby effectively sealing the weakened cavity. Accordingly, this may simplify use of the device, and reduce the complexity of the surgery required to install the hernia plug device in the hernia affected region.

The disclosed smart 3D hernia plug devices also achieves a number of other appreciated benefits, including practicality, ease of use, low cost of production as well as provides easy modification to accommodate people of all ages, genders, and sizes.

### I. General Overview

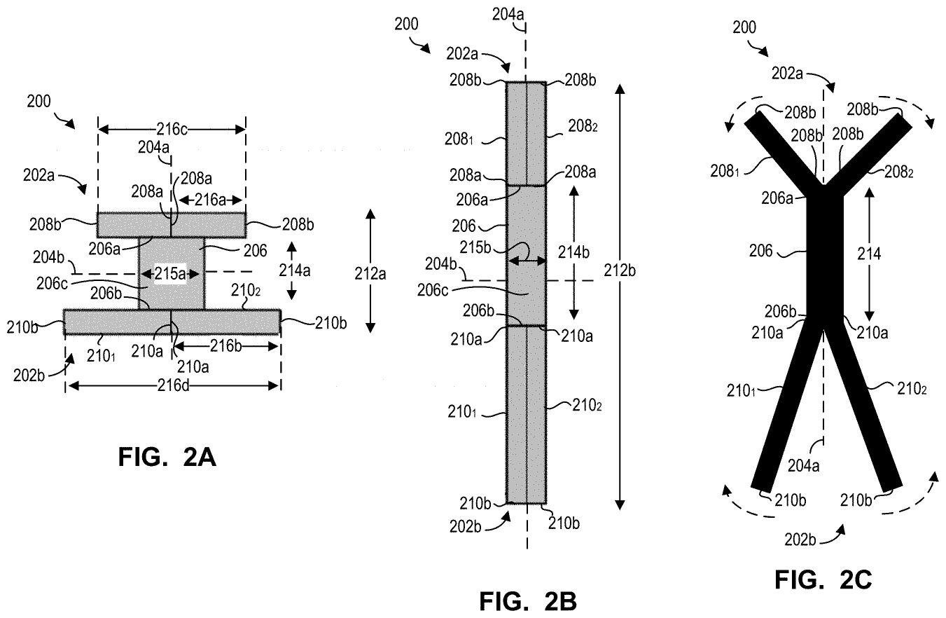

With reference now to FIGS. 2A-2C, a general description of the smart 3D hernia plug is now provided. FIGS. 2A-2C show simplified illustrations of a two-dimensional (2D) projection of a smart 3D hernia plug 200, in accordance with embodiments provided herein.

As previously described, in accordance with the teachings herein, the disclosed hernia plug 200 may be manufactured from smart material (e.g., a smart polymer material (SMP)). This, in turn, enables the plug to transform between a compressed, pre-activation state (FIG. 2B) and an un-compressed, post-activation state (FIG. 2A).

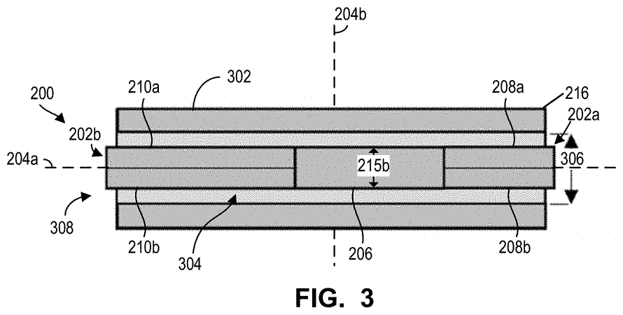

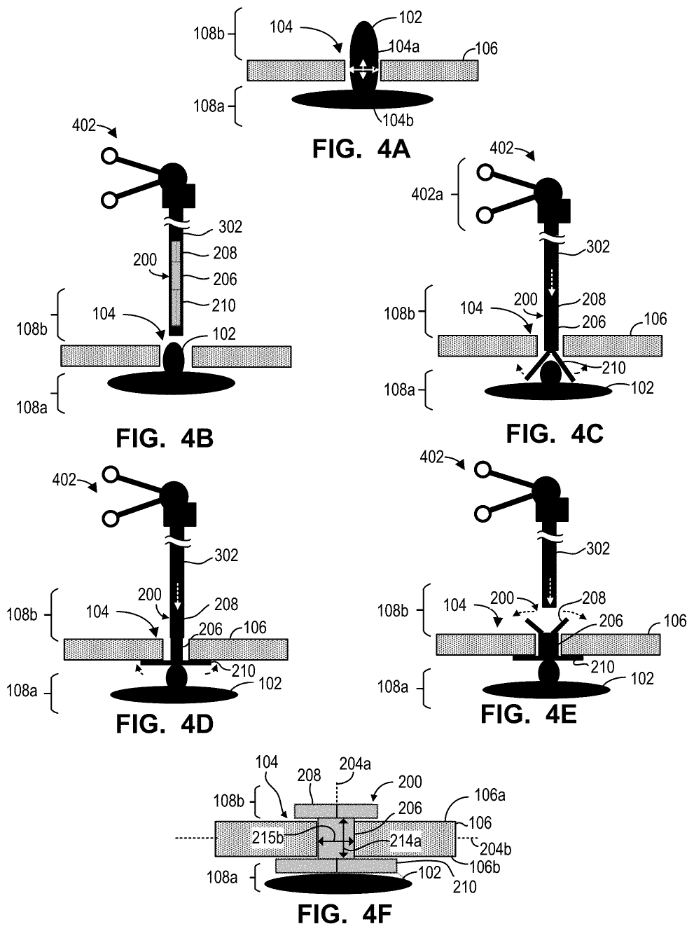

In the compressed, pre-activation state (FIG. 2B), the hernia plug 200 is adapted for easy transport to a hernia site. For example, the plug may be transported in the pre-activated state by a conveyance device (e.g., a laparoscope) (FIGS. 3, and 4A-4E). At the hernia site, and in response to an activation trigger (also known as an activation stimuli), the hernia plug 200 may transform into the expanded, post-activation state (FIG. 2A). In the post-activated state, the hernia plug may expand to effectively block or seal the hernia site (FIG. 4E).

To facilitate transition of the hernia plug 200 between the pre- and post-activated states, the plug 200 is manufactured from a shape-memory polymer (SMP) which ‘activates’ under thermal stimuli (e.g., a thermo-responsive SMP). The SMP may be adapted to have a glass transition temperature (T_{g}) (e.g., 150a in FIG. 1D) generally corresponding to the internal human body temperature. As such, at the hernia site, the smart hernia plug 200 may automatically activate, or deploy, in response to body generated heat.

In other cases, the plug may be manufactured from an SMP that is adapted to activate under any other type of stimuli (e.g., chemical, radiation, ultraviolet (UV), etc.) In still other cases, rather than being manufactured from an SMP, the plug may be manufactured from a shape-memory alloy (SMA) that similarly activates under any type of desired stimuli and operates under similar theoretical principles (e.g., nitninol). That is, the plug can be broadly manufactured from any materials (or combination of materials) that enable the plug to change between a compressed and expanded state shape in response to one or more stimuli. For example, the plug may be manufactured from a class of smart memory materials SMMs (e.g., SMPs, SMAs, etc) having the ability to recover their original shape from a deformation when a particular stimulus is applied (e.g., exhibiting shape-memory effect).

In still yet other cases, the plug may be manufactured from a change-shaping flexible material that does not necessarily require a stimulus (e.g., rubber). Accordingly, the plug can broadly change, in one manner or the other, between a compressed and an expanded shape states. For ease of description, the remainder discussion will focus on an example embodiment where the hernia plug is manufactured from a thermo-responsive SMP.

Now in more detail, referring still to FIGS. 2A-2C, the smart hernia plug 200 extends between a first end 202a and a second end 202b. The ends 202a, 202b may be axially opposed along a longitudinal extension axis 204a. A length dimension 212 is defined between the first and second ends 202a, 202b, and along axis 204a. In at least one embodiment, the length dimension 212a, in the post-activated state (FIG. 2A) is smaller than the length dimension 212b in the pre-activated state (FIG. 2B).

As further exemplified, the hernia plug 200 is generally formed of one or more of the following: (i) a waist portion 206; (ii) one or more first overhangs 208; and (iii) one or more second overhangs 210.

Waist portion 204 defines a mid-body segment of the plug 200 and extends, along the longitudinal axis 204a, between a first waist end 206a and an axially opposed second waist end 206b. The first waist end 206a is proximal the first plug end 202a, while the second waist end 206b is proximal the second plug end 202b. A waist length 214 is defined, along axis 204a, between the first and second waist ends 206a, 206b.

A waist width dimension 215 is also defined along a lateral axis 204b. Lateral axis 204b extends orthogonally to the longitudinal axis 204a. As noted previously, FIG. 2 merely represents the 3D plug as a two-dimensional projection. According, observed in three dimensions, the width dimension 215 may in-actuality correspond, for example, to a diameter of the waist portion 206.

In at least one embodiment, the waist width 215 can expand in the post-activated state. That is, the post-activated waist width 215a (FIG. 2A) is greater than the pre-activated waist width 215b (FIG. 2B). As explained herein, this can enable the waist to expand to effectively seal a hernia affected site 104 in the post-activated state (FIGS. 4C and 4F).

Turning now to the first and second overhangs 208, 210—as exemplified, each of the overhang(s) 208, 210 is movably coupled to the first and second waist ends 206a, 206b, respectively. For example, first overhang(s) 208 are movably coupled to the first waist end 206a, while the second overhang(s) are movably coupled to the second waist end 206b.

In an upright position, the first overhangs 208 are axially positioned (e.g., along axis 204a) vertically above the second overhangs 210. Accordingly, in at least some embodiments, the first overhangs 208 are also referenced herein as the “top” overhangs 208, while the second overhangs 210 are referenced herein as the “bottom” overhangs 210. It will be understood, however, that plug 200 is not so limited to any particular orientational configuration.

Each of the first and second overhangs 208, 210 can comprise one or more longitudinal members. Each longitudinal member extends between a respective first overhang end 208a, 210a and a respective second overhang end 208b, 210b. As exemplified, the first ends 208a, 210a—of each overhang—are movably coupled to the respective waist end 206a, 206b.

As further exemplified, in the pre-activation state (FIG. 2B), the overhangs 208, 210 are in a generally contracted position. In the contracted position, overhangs 208, 210 extend substantially parallel to the longitudinal axis 204a (e.g., within ±30° of the longitudinal axis 204a). In the post-activation state (FIG. 2A), overhangs 208, 210 may translate into a generally expanded position, e.g., in response to an activation trigger. In this expanded position, overhangs 208, 210 extend substantially parallel to the lateral axis 204b, (e.g., within ±30° of the width axis 204b), or generally orthogonally to the longitudinal axis 204a.

In at least one embodiment, overhangs 208, 210 may rotate outwardly to translate between the pre-activation and the post-activation states (FIG. 2C). As provided herein, in the post-activation state (FIG. 2B), the function of the overhangs 208, 210, is to secure the hernia plug 200 to the hernia-affected site.

Reference is now made concurrently to FIGS. 3 and 4A-4F, which are provided to further demonstrate an example application of the smart 3D hernia plug 200.

As explained above, the ability of the hernia plug 200 to transform between the pre-activation state (FIG. 2B) and the post-activation state (FIG. 2A) can facilitate ease of transportation and implantation of the plug 200 at a hernia site.

In respect of the pre-activation state: the smart plug may be received inside of a conveyance device. The conveyance device can transport the hernia plug 200 to the hernia affected site. For instance, as best exemplified in FIG. 3, the smart hernia plug 200 may be designed to be received inside of the hollow interior 304 of a cylindrical tube 302, e.g., a tube extending along longitudinal axis 204a. The cylindrical tube 302 may be associated with a conveyance device, such as a laparoscope. In this manner, the laparoscope is insertable into the human body, and used to transfer (or carry) the hernia plug 200 to the target hernia site (e.g., 104 of FIG. 4A).

It will now be understood, with reference to FIG. 3, that the parallel, compressed configuration of the overhangs 208, 210—in the pre-activated state—is such as to enable the plug 200 to be received inside the elongated cylinder interior 304. Additionally, the reduced pre-activation width 215b is also to facilitate accommodation of the plug 200 inside the elongated tube 302, e.g., the waist width 215b may be smaller than the diameter 306 of the tube 302. While laparoscopes are available in a variety of sizes including but not limited to 3 mm, 5 mm, and 10 mm in diameter 306, the 10 mm size is widely used for hernia repair surgeries. Accordingly, in at least one embodiment, the smart plug 200 may have a pre-activated waist width 215b of approximately 10 mm.

Referring now to FIG. 4A-4F, in the pre-activated state, the hernia plug 200 may be transportable to the hernia site 104, via a conveyance device 402 (e.g., a laparoscope) (FIG. 4B). Once transported, the hernia plug 200 is implanted at the site 104. For example, the hernia plug 200 may be “pushed out” of the laparoscope tube 402, via the laparoscopic handle 402a (FIG. 4C). Upon deployment, the hernia plug 200 may initiate automatic transformation from the pre-activated state into the post-activated state. For example, this transformation can occur automatically in response to thermal stimuli. The thermal stimuli can correspond, for example, to the body's internal heat. The thermal stimuli causes the plug's thermo-responsive shape-memory polymer (SMP) to activate, and in turn expand into the post-activated state (FIGS. 4C-4F).

In more detail, in repairing inguinal hernia, the conveyance device (e.g., the laparoscope tube 402) may be positioned to first deploy the second overhangs 210 into the abdomen-side 108a. For example, the opening of the laparoscope tube 308 (FIG. 3) may be positioned proximal the abdomen side 108a. The hernia plug 200 may also be positioned, within the laparoscope tube, such that the second overhangs 210 are first to exit the opening 308.

Accordingly, the second overhangs 210 initially expand to engage the inner abdomen-side in the post-activated. In other words, the second overhangs 210 may extend (e.g. rotate) to substantially align with the lateral axis 204b, such as to engage the abdomen-side 108a of the abdominal wall 106 (FIGS. 4C, 4D). In expanding, the second overhangs 210 may also push the protruding organ 102 back into the patient's abdominal area.

The conveyance device 402 may then be operated to deploy the waist portion 206 at the hernia site 104 (FIGS. 4D, 4E). That is, the conveyance device 402 may be positioned to deploy the waist portion 206 such that the waist 206 extends through the abdominal wall 106, as between the abdomen side 108a and the groin side 108b.

In the process of being deployed, the waist portion 206 may also expand such that the post-activated waist width 215a effectively seals the hernia site 104 (FIG. 4F). In at least one embodiment, the waist 206 may have a generally circular cross-sectional profile, such that in the post-activated state, the waist 206 radially expands along lateral axis 204b to engage the edges of the abdominal wall 106 (FIG. 4F). That is, in the post-activated, the waist width 215a (or diameter) expands to be substantially equal to the width 104a (or diameter) of the hernia defect 104, e.g., along width axis 204a (FIG. 4A).

In some embodiments, the post-activated waist width 215a may be approximately 50 millimeters. In other cases, it can vary between a range of between 40 mm to 60 mm. The waist length 214a, in the posted activated state, may also be substantially equal to the height 104b of the hernia defect 104.

Once the waist 206 is deployed, the first overhangs 208 may then be subsequently deployed above the abdominal wall 106, e.g., on the groin-side 108b (FIG. 4E). In the post-activated state, the first overhangs 208 also respond to the thermal stimuli to expand (e.g., rotate) to engage the groin-side of the abdominal wall 106. In other words, the first overhangs 208 may rotate to extend along lateral axis 204b so as to engage the groin-side 108b of the abdominal wall 106 (FIG. 4F).

In the deployed and post-activated state (FIG. 4F)—the hernia plug 200 is uniquely adapted be substantially self-gripping. That is, the hernia plug 200 is able to positionally lock in place, thereby effectively blocking the organ 102 from re-exiting the abdomen area 108a.

The self-gripping, or self-locking feature, is realized through the combination of one or more of: (i) the one or more first overhangs 208 that face and engage the outer-side of the abdominal wall 106a (e.g., the groin-side); (ii) the one or more second overhangs 208 that face and engage the inner-side of the abdominal wall 106b (e.g., the abdominal-side) and (iii) the central waist 206 that expands in the post-activated state to fill the volume of the hernia gap 104.

Therefore, in this expanded configuration, the abdominal wall 106 is disposed between first and second overhangs 208, 210, and motion of the hernia plug 200 is minimized both in the longitudinal direction (e.g., along axis 204a), as well as in the lateral direction (e.g., along axis 204b).

With reference to FIG. 2A, in at least one embodiment, due to the availability of space on the abdomen-side 108a of the wall 106—the second overhangs 210 can be designed to be slightly longer than the first overhangs 208. Accordingly, in the post-activated state, each of the second overhangs 210 may have a larger length 216b then the length 216a of the first overhangs 208. As such, in accordance with at least some embodiments, the first overhangs 208 may also be referred to herein interchangeably as the short overhangs 208, while the second overhangs 210 may be referred to herein interchangeably as the long overhangs 210.

In one example case, the first overhangs 208 may each have a length 216a of approximately 15 mm (or a total end-to-end length 216c of 30 mm), while the second overhangs 210 may each have a length of approximately 25 mm (or a total length end-to-end 216d of 50 mm). The inventors have appreciated, through trial and error, that these dimensions are optimal for hernia repair.

A number of advantages will now be appreciated with respect to the design configuration of the smart 3D hernia plug 200.

First, as the plug 200 does not rely on tension-based repair (e.g., suturing the weakened tissue together), use of the plug 200 reduces various complications that include tearing, bleeding, hematoma as well as nerve entrapment, which ordinarily cause poor tissue regrowth. This, in turn, reduces patient discomfort and pain.

Second, plug 200 is configured to be self-gripping (or self-locking). Accordingly, contrary to existing 2D or 3D meshes, the plug does not require external securing mechanisms to anchor the plug in-place. As such, this minimizes foreign body reactions ordinarily caused by the use of sutures, tacks or adhesive, and which typically contribute to complication rates in hernia repair procedures using ordinary 2D or 3D meshes.

Third, similar to other 3D meshes, the plug 200 displays superior compliance to dynamic motion. In turn, contrary to static meshes, the 3D plug design can accommodate increased motility, e.g., of the abdominal wall.

Fourth, contrary to existing 3D mesh solutions, the disclosed smart 3D plug does not require excessive operating time and procedure complexity. More particularly, the operating time and complexity is minimized by virtue of the plug being easily transportable to the hernia site, in the pre-activated state, using common surgical tools (e.g., a laparoscope), and further, its automatic deployment into the post-activated state upon implanting at the hernia site. Owing to the self-deploying capabilities, the 3D hernia plug is distinguished from conventional meshes which require intricate surgical precision and accuracy to ensure complete placement of the mesh at the hernia site. Further, procedure time and complexity is reduced by virtue of the mesh's self-gripping nature. In particular, as stated previously, the operating surgeon is not required to invest time in applying an external securing mechanism to the plug to secure it in place (e.g., by suturing the plug to the tissue).

Other advantages of the disclosed 3D hernia plug will now also occur to the ordinary skilled artesian.

### II. Example Embodiments of Smart Three-Dimensional (3D) Hernia Plug

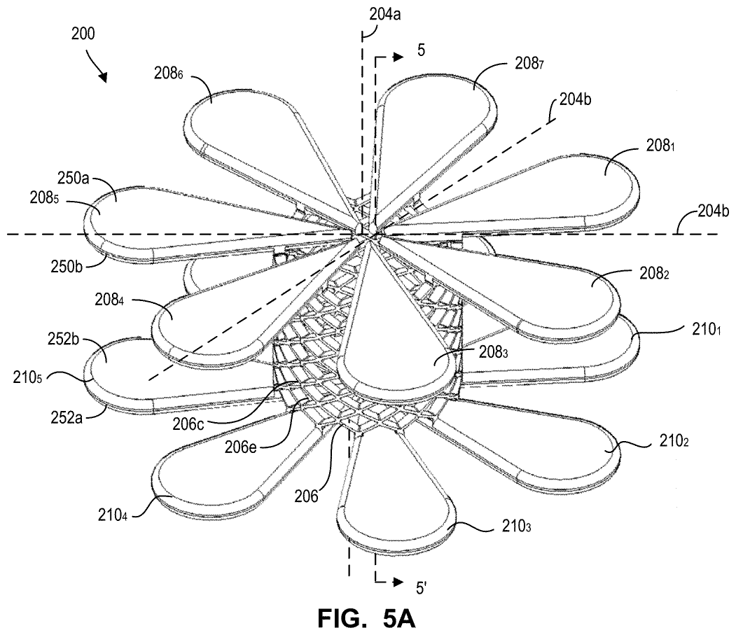

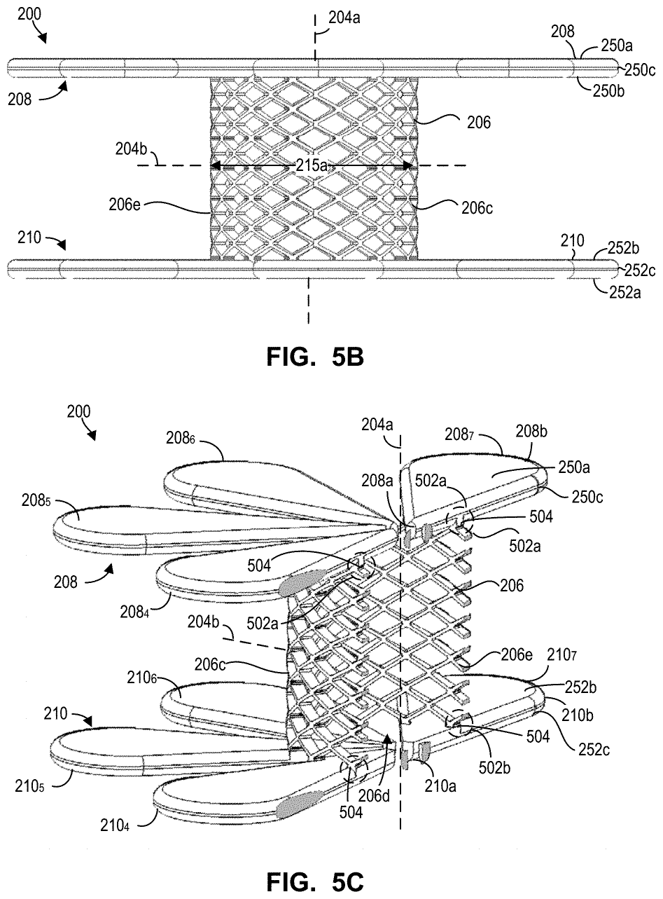

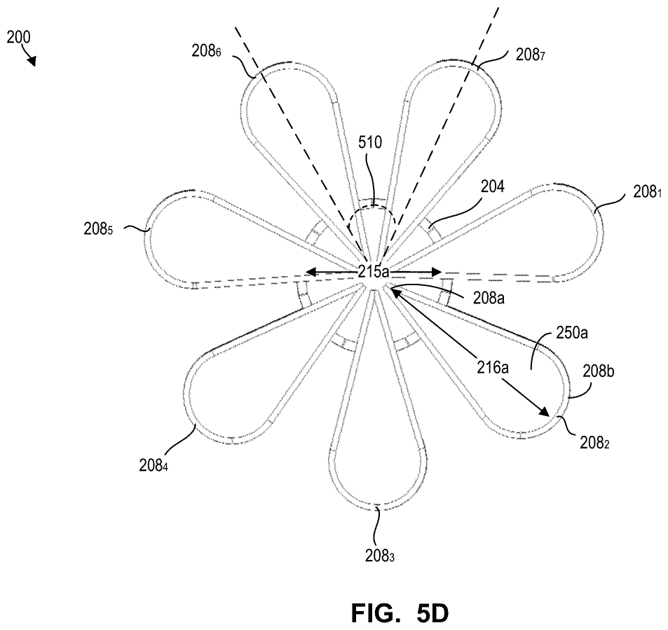

Reference is now made to FIGS. 5-9, which illustrate various example embodiments for configurations of the smart hernia plug 200. These figures exemplify various example design possibilities for the waist portion 206, and the first and second overhangs 208, 210.

It will be understood that while various features are separately illustrated—the hernia plug can include any combination of illustrated features, alone or in combination. For example, various illustrated overhang and waist designs can be combined into a single hernia plug, even though these designs not explicitly illustrated together in the figures.

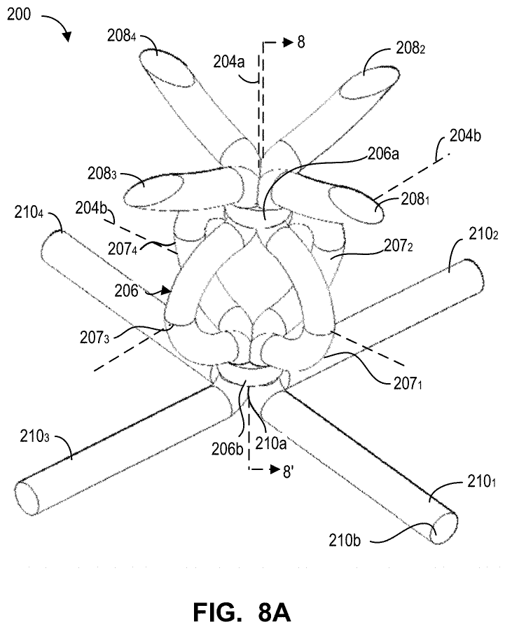

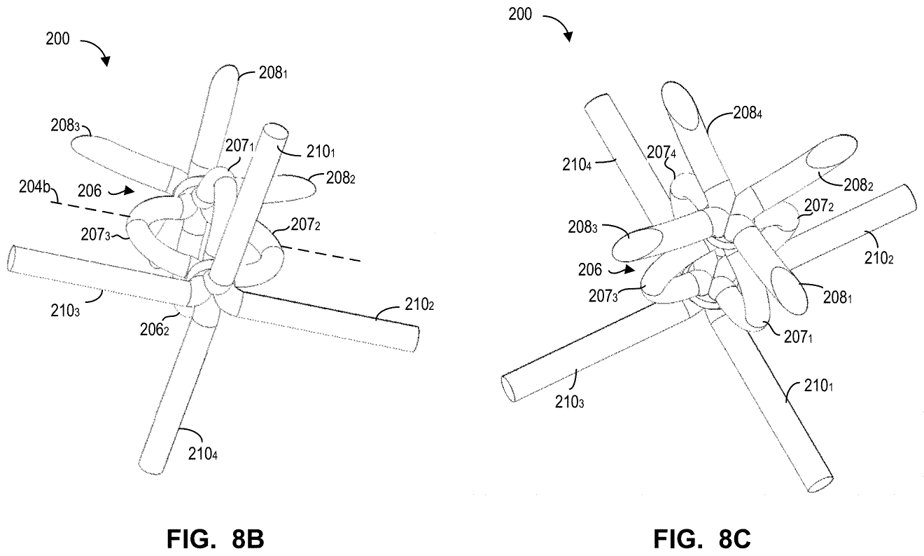

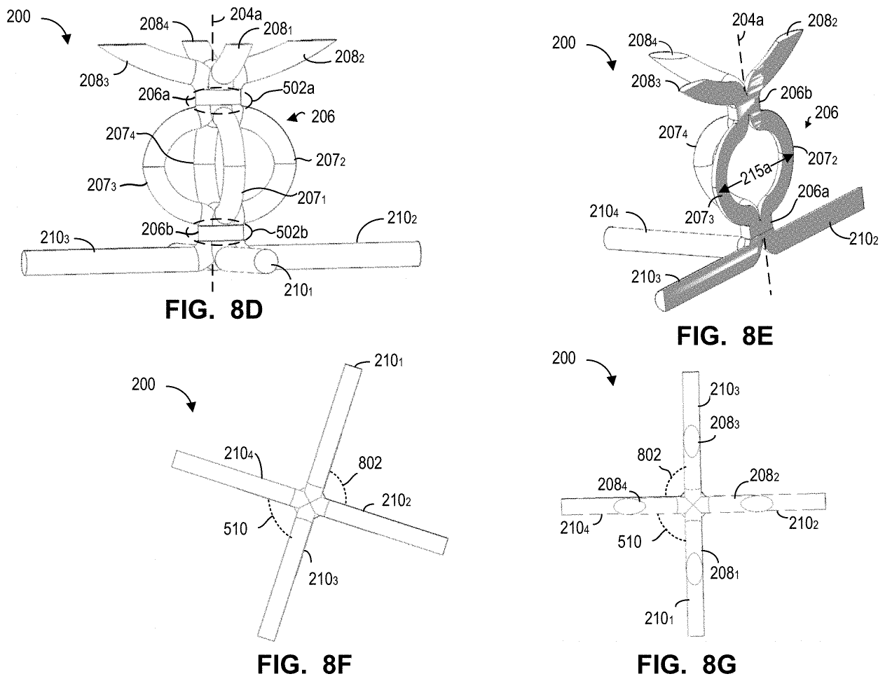

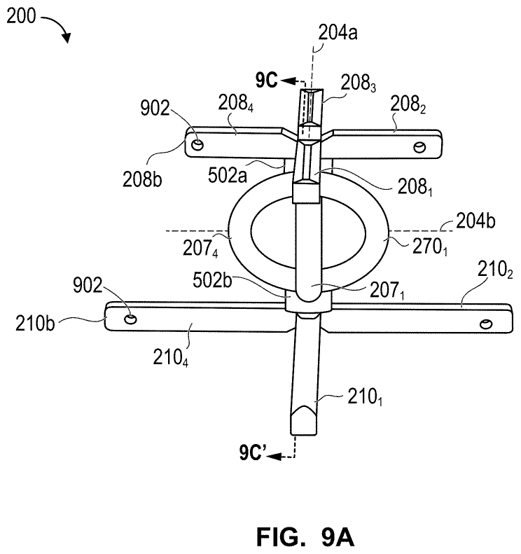

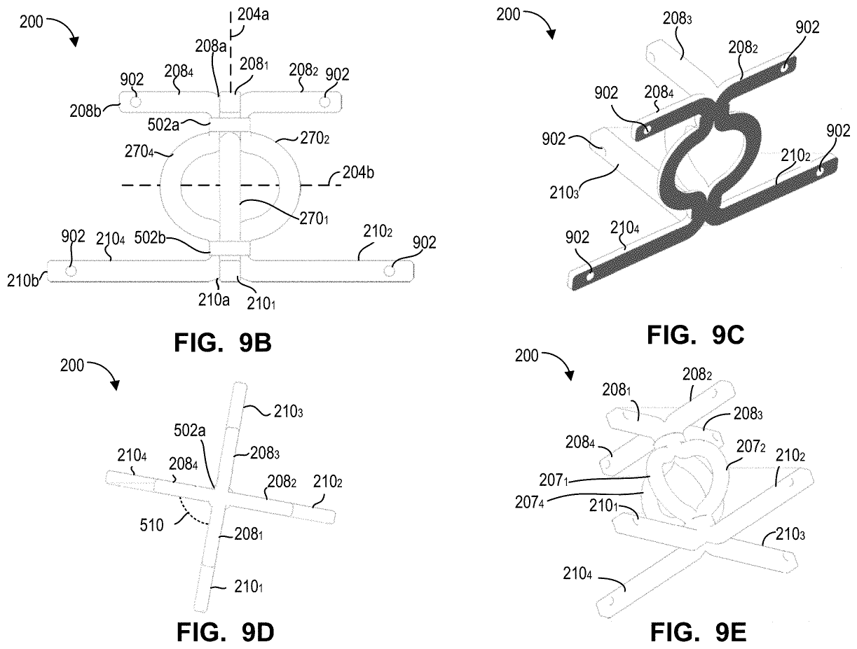

As shown the waist portion 206 may have a number of design and shape configurations. For instance, as exemplified in FIGS. 5-7, the waist portion 206 may comprise one or more connected continuous members, extending between the first waist end 206a and the second waist end 206b. In other embodiments, best exemplified in FIGS. 8-9, the waist portion 206 may comprise one or more discrete members 207₁—207₄, also extending between the first waist end 206a and the second waist end 206b.

In embodiments where the waist portion 206 comprises continuous members (FIGS. 5-7), the waist member may have a lateral surface 206c. Further, the continuous waist member may be variably configured. For instance, as exemplified, the waist portion 206 may have a cylindrical shape with a generally circular cross-sectional profile, defined in a plane orthogonal to the longitudinal axis 204a. Accordingly, in the post-activated state, the waist 206 may expand radially outwardly, along an axis orthogonal to the longitudinal axis 204a, to achieve an expanded cross-sectional diameter 215a (FIGS. 5B, 5D, 6B, 6E, 7C, 7E). In this manner, using a circular cross-sectional profile, the waist 206 is able expand uniformly radially outwardly in each direction so as to more effectively to block the hernia defect region 104. In other cases, the waist 206 may have any other suitable cross-sectional design, e.g., square, rectangular, triangular etc.

Preferably, the continuous waist member 206 may have a hollowed interior 206d (FIG. 5C, 6C, 7C). The hollowed interior may decrease the overall weight of the plug. As noted previously, lightweight meshes have the advantage of minimizing foreign body reactions, and thereby decreasing the body's inflammatory response. The hollowed interior can also increase the flexibility of the waist portion 206, such that the waist may quickly transition between the pre- and post- activated states, e.g., as there is less material which is expanding and contracting. Additionally, the hollowed interior 206d simplifies the design complexity, and reduces material manufacturing costs. In other embodiments, however, the continuous waist member 206 may have only a partially-hollowed or non-hollowed interior.

In some embodiments the continuous waist member 206 may have a plurality of perforations 206e. The perforated surface may further enhance the flexibility of the waist portion 206 to facilitate transition of the waist between the pre- and post-activated state. Perforations 206e may also further minimize the weight of the plug such as to also reduce foreign body reaction. Still further, perforations 206e can reduce the volume of material required to fabricate the waist 206, thereby further reducing the design complexity and manufacturing cost.

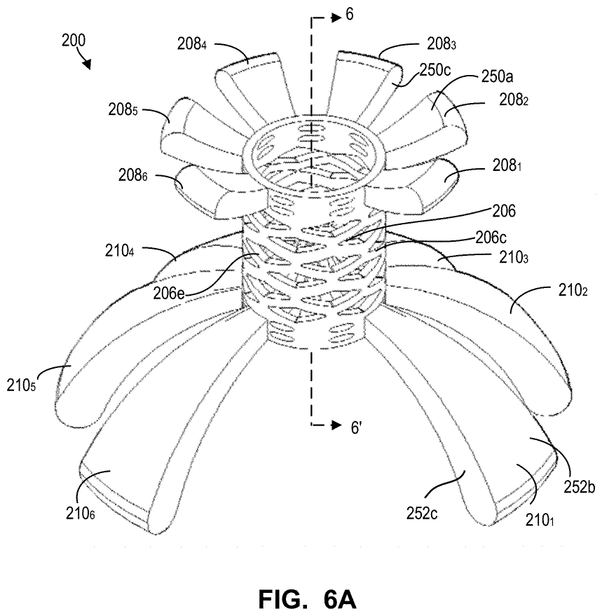

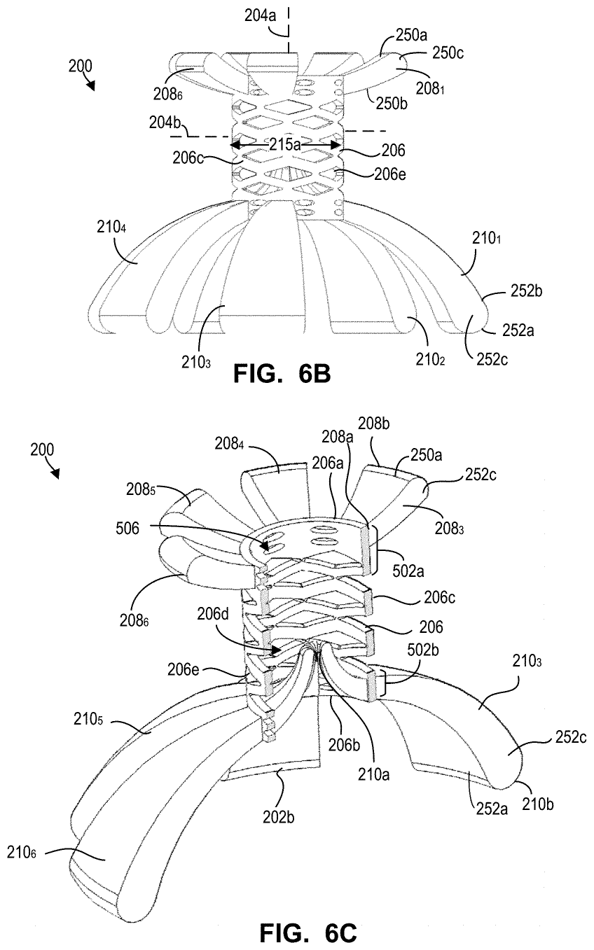

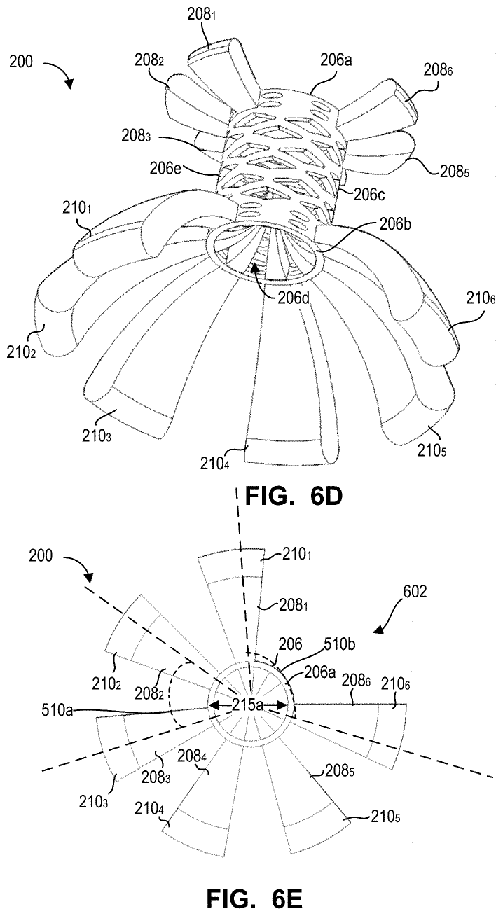

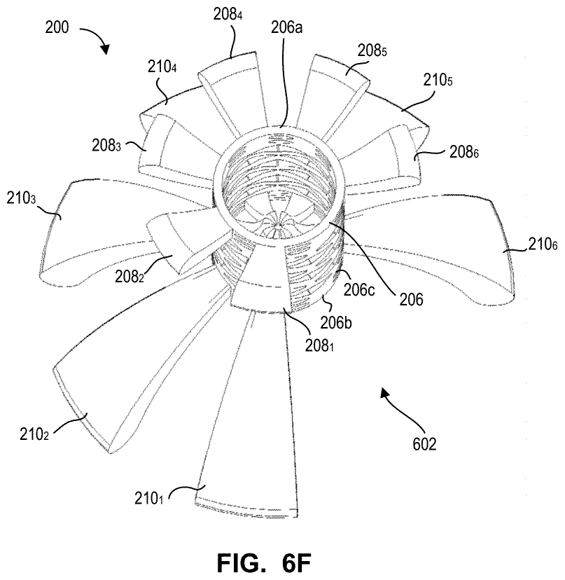

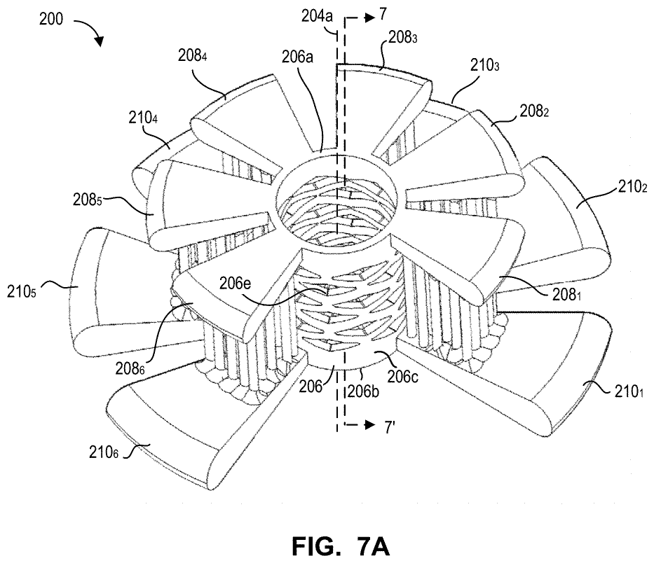

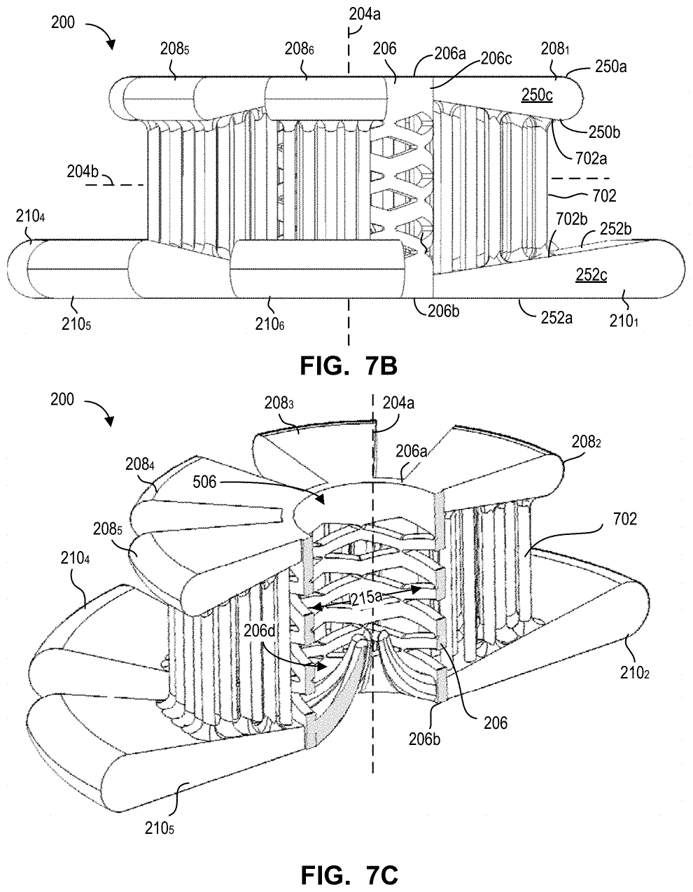

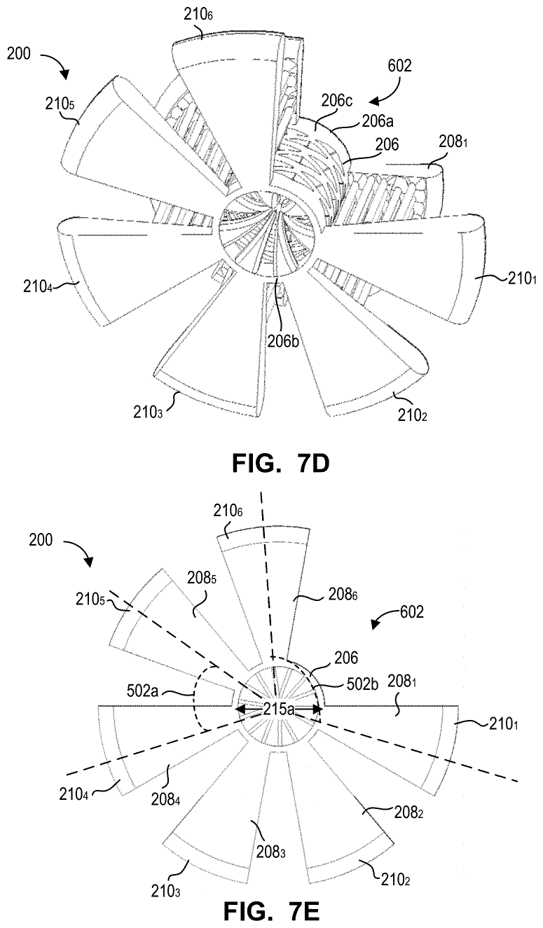

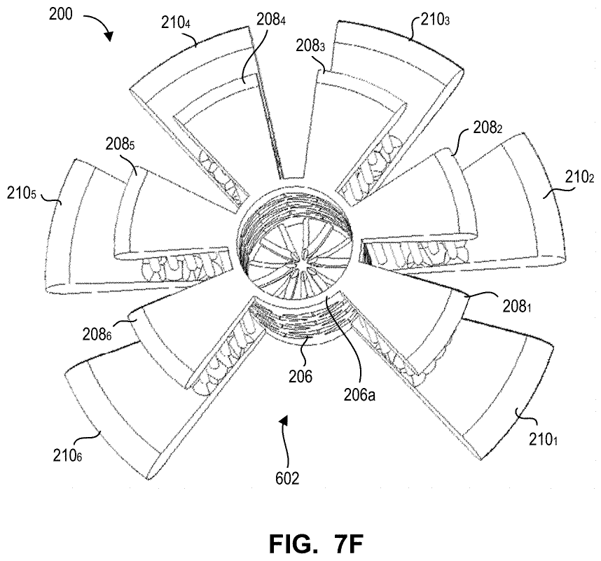

Perforations 206e may cover any suitable portion of the total surface area of the waist's lateral surface 206e. For example, perforations 206e may cover a substantial portion of the waist's lateral surface 206c (FIGS. 5A-5D) (e.g., greater than 75% of the surface area). In other cases, the perforations 206e may be more interspersed such as to cover only a portion of the waist's lateral surface 206c (FIGS. 6-7) (e.g., greater than 50% of the surface area). In other cases, the perforations 206e may cover any percentage of surface area—for example, perforations 206e can cover 5% to 90% of the total surface area of the waist's lateral surface 206c.

Perforations can also have any suitable shape. In the exemplified embodiment, the preformation shape is a diamond like shape (FIGS. 5 and 6) to facilitate expansion and compression of the waist 206 along the longitudinal and lateral axis directions 204a, 204b. In other cases, the shape can be elliptical or circular, by way of non-limiting example.

In other embodiments, as exemplified in FIGS. 8-9, the waist portion 206 may comprise one or more discrete elongated members 207₁-207₄. Each discrete member 207 may extend between the first waist end 206a and the second waist end 206b. In the pre-activated state, the elongate members 207₁-207₄ may be substantially parallel to each other, along the longitudinal axis 204a. In the post-activated state, a portion of each elongate member 207—e.g., a portion between the first and second end 206a, 206b—may expand radially outwardly in a direction orthogonal to the longitudinal axis 204a (FIG. 8E). An advantage of using elongate members 207 for the waist is explained in greater detail herein.

As exemplified in FIGS. 8-9, any number of elongated waist members 207 may be provided, and in any suitable positional arrangement. In the exemplified embodiment, the waist portion 204 comprises four elongated members (207₁-207₄), each equally spatially spaced by an angle 802 (FIG. 8F) of 90°. Angle 802 is defined in a plane perpendicular to the longitudinal axis 204a. The use of four elongate members, equally spaced, provides increased structural integrity in each radial direction. Further, as provided herein, in embodiments where the plug is used for treating inguinal hernia defects, the large spacing between the elongate members 207 may accommodate the spermatic cord passing through the plug.

Similar to the waist portion 206, overhangs 208, 210 may also have any number of possible shapes and design configurations. For example, overhangs 208, 210 may have a substantially planar shape (FIGS. 5-7), or may comprise rod-like elongate members (FIGS. 8-9).

In more detail, as best exemplified in FIGS. 5B, 6B and 7B, in at least some embodiments—overhangs 208, 210 can have a substantially planar design. For example, each overhang 208, 210 may have a respective outer surface 250a, 252a, and a respective inner surface 250b, 252b. Each of the surfaces 250, 252 may have a respective planar surface area.

As used herein, outer surfaces 250a, 252a refer to the set of overhang surfaces 208, 210 directed away from each other, e.g., along axis 204a. Further, inner surfaces 250b, 252b refer to the set of overhang surfaces directed towards each other, e.g. along axis 204a. Accordingly, the inner and outer surfaces, on each overhang, are axially opposed, e.g., along axis 204a. Each of the overhangs 208, 210 may also have one or more respective lateral surfaces 250c, 252c. Lateral surfaces 250c, 252c extend between the respective first and second surfaces 250, 252.

An advantage of the planar overhang design (FIGS. 5-7) is that the surface area—of each of the first and second surfaces 250, 252—enhances the stability of the hernia plug when deployed to the hernia affected site. That is, as exemplified in FIG. 4F, the large inner surface area 250b, 252b, of each overhang, provides a larger contact area for engaging with the abdominal wall 106. This, in turn, better secures the hernia plug in-place, within the hernia affected region.

In some cases, as exemplified in FIG. 6, the planar overhang designs can also have slight curvature or arching, which can facilitate printing of the overhangs.

In some cases, the planar overhangs can have a tapering design. That is, each overhang can have wider profile at second end 208b, 210b, as compared to the first end 208a, 210. This can be observed in the embodiment of FIG. 5, whereby each overhang has a petal-like design. An advantage of this design is that the narrower first end 208a, 210a facilitates folding of the overhangs in the pre-activation state. Further, the wide second end 208b, 210b can provide for more surface area contact inside the body to prevent movement or displacement of the hernia plug therein.

In other embodiments, best exemplified in FIGS. 8-9, each of the overhangs 208, 210 can comprise a rode-like elongate member. An advantage of this configuration, is that the overhangs 208, 210 have a reduced size and mass. This can improve shape memory programming and recovery of the hernia plug 200, as provided in further detail herein.

In still yet other embodiments, rather than comprising discrete members—each of the overhangs 208, 210 may comprise a single continuous member (or one or more connected continuous members). For example, each of the overhangs 208, 210 may comprise one or more connected continuous members that surround at least a portion of the waist 206. The one or more connected continuous members may comprise a perforated or un-perforated surface.

Irrespective of the design configuration selected for the overhangs 208, 210—the overhangs 208, 210 may have any suitable positional configuration in the post-activated state.

For example, as best exemplified in FIGS. 5D, 8F and 8G, the overhangs 208, 210 may be equally spaced, symmetrically around the circumference of the waist 206. That is, adjacent first overhangs 208 may be equally spaced in a plane orthogonal to the longitudinal axis 204a. Similarly, adjacent second overhangs 210 may also be equally spaced along a plan orthogonal to the axis 204a. In other words, the angle 510 between adjacent overhangs 208, 210 may be equal. The symmetric design may be useful, in some cases, for navigating the plug to the desired hernia location. Further, the symmetric design may enable quick (or easy) deployment. For instance, a medical practitioner (e.g., surgeon) may simply deploy the symmetric design without concern about configuring and re-positioning the plug to fit inside the hernia location owing to some degree of asymmetry in the design.

In other embodiments, the spacing between adjacent overhangs 208, 210 may not be necessarily equal. For example, as exemplified in FIGS. 6E and 7E, at least two adjacent overhangs are more widely spaced apart, e.g., by a gap 602, than the remaining overhangs. For example, in FIG. 6E, each of the overhangs 208₁ and 208₂, 208₂ and 208₃, 208₄ and 208₅, and 208₅ and 208₆ are equally spaced apart by an angle 510a. However, overhangs 208₁ and 208₆ are spaced apart by a larger angle 510b. A similar configuration is adapted for the second overhangs 210₁ and 210₆. An advantage of this configuration is that, for inguinal hernia applications, the gap 602 may accommodate a spermatic cord passing through the hernia plug 200 and without obstruction. In at least one embodiment, the gap 602 may accommodate a spermatic cord within a 11 to 26 mm in diameter.

The first and second overhangs 208, 210 may also be arranged in any suitable position configuration with respect to each other. For instance, the first and second overhangs can be arranged in pairs aligned along longitudinal axis 204a. For example, in FIG. 5A, overhangs 208₁ and 210₁ are parallel along axis 204a, and so forth for 208₂ and 210₂ and so on. This, in turn, may also provide greater symmetry to the design, as previously discussed.

In the embodiment of FIGS. 8-9, the elongate waist members 207 may also be aligned with respective first and second overhangs 208, 210. For example, waist member 207₁ may be aligned with overhangs 208₁ and 210₁ along an axis parallel to axis 204a, and so forth.

Various possible design configurations can also be used to adjoin the overhangs 208, 210 to the waist portion 206.

In the exemplified embodiments, waist portion 206 may have one or more connection portions 502. Connections portions 502 may define locations at which the waist 206 may connect to the first and second overhangs 208, 210. For instance, the waist portion 206 may have a first connection portion 502a for connecting to the first overhangs 208, and a second connection portion 502b for connecting to the second overhangs 210 (FIGS. 5C, 6C, 7C and 8D).

As best exemplified in FIGS. 5C and 8D, in at least one embodiment, the first connection portion 502a may be located directly at the first waist end 206a. Further, the second connection portion 502b may be located directly at the second waist end 206b. In the embodiment of FIG. 5C, the first and second connection portions 502a are defined directly along waist edge, and overhangs 208, 210 are connected to the waist 206 at one or more engagement points 504 defined along the edge.

In other embodiments, best exemplified in FIGS. 6C and 7C, connection portions 502a, 502b define a segment of the lateral waist surface 206c, e.g. proximal the first and second waist end 206a, 206b, respectively. In these embodiments, connection portions 502a, 502b may comprise a substantially solid ring of material. An advantage of this configuration is to further enhance the structural integrity of the connection between the waist portion 206 and the overhangs 208, 210, e.g., to allow the overhangs to better translate between the pre-activation state and the post-activation state.

In some embodiments (FIGS. 6C, 7C and 8D), the overhangs are connected to the respective connection portion via the respective first overhang end 208a, 210a. An advantage of this configuration is that, in the post-activated state, the overhangs are able to extend radially outwardly to fully engage the abdominal wall 106 (FIG. 4F) using the entire length of the overhangs.

In other embodiments (FIG. 5C), the overhangs 208, 210 engage the connection portion 502a, 502b at a mid-portion of the overhangs, e.g., a mid-portion defined between the respective first and second overhang ends 208a, 208b and 210a, 210, 210b. This design may increase the structural integrity of the connection between the overhangs and the waist portion. For example, by connecting the overhangs at the midportion, a fulcrum-like point is generated which can help manage the weight of the overhang, as well as preventing breaking of the overhangs. Accordingly, the overhangs 208, 210 may better translate between the pre- and post-activation state. Additionally, the extension of the overhangs into the waist portion 206 can act to prevent (or block) the protrusion of the hernia through the center of the waist portion 206.

As best exemplified in FIGS. 6C, 7C, different connection configurations can be used in the same plug. For instance, the first overhangs 208 may connect at their respective first end 208a to the waist connection portion 502a. In contrast, the second overhangs 210 may connect to the waist connection portion 502b at their mid-portion. This design may accommodate the fact that the first overhangs 208 are shorter in length than the second overhangs 210. Accordingly, the second overhangs 210 may benefit structurally from being connected at their mid-portion.

Reference is now made more specifically to the hernia plug 200 embodiment exemplified in FIGS. 9A-9E, which is described in greater detail herein.

In particular, as compared to the other exemplified embodiments, the embodiment in FIG. 9 is believed to demonstrate a number of unique features, including: (i) an improved overall surface finish of both the first and second overhangs 208, 210, (ii) higher printability and repeatability in terms of fabrication, and (iii) ease of translation between the post- and pre-activated state, with minimal damage to the structure of the plug.

To this end, as stated previously, the use of rod-shaped overhangs 208, 210, as well as the elongated waist members 207 (e.g., rather than the continuous waist member design), allows for a more simplified, and less large and bulky design. This in turn, facilitates faster shape memory programming and recovery, with minimized damage to the structure of the plug during transition between the pre- and post- activated states. That is, using less material and a simplified design—the transition of the plug between the pre- and post- activated state is also simplified as less material and structural components are required to undergo the transition (e.g., expand or contract). For example, using discrete waist members 207, it is easier to alter the diameter of the waist portion 206 as compared to using the continuous waist design, which is more difficult to contract and expand in diameter and length.

Further, the design of the connection areas 502a, 502b in this embodiment assists in printability, by providing a solid base for the waist 206 to be printed on. The use of the solid connection areas 502a, 502b disposed on either end of the waist 206, also provides stronger structural support for the waist members 207 as they expand and contract during transition between the pre- and post-activated states. In particular, by providing this structural support, this design minimizes deformation of the waist portion 206 during the transition of the plug between the pre- and post-activated states. This is contrasted to some of the other designs (e.g., in FIGS. 6-7) which do not similarly provide a solid base portion, but rather the connection portions 502 just forms a side surface 206c of the waist and therefore itself alters in diameter during shape transition of the plug.

In at least one embodiment, in the hernia plug 200 in the embodiment of FIG. 9—the short overhangs 208 in the post-activated state have a span of 30 mm and the long overhangs 210 have a span of 50 mm. The angle between the overhangs 510 is 90° so as to allow for the spermatic cord to pass. The waist 206 is able to expand to a span of 20 mm in the post-activated state with the post-activated height 212a of the plug 200, from long to short overhang, being 27 mm. Further, the long overhangs 210 may span 50 mm in diameter 216a, in the post-activated state, which assists in gripping position at the hernia-site.

In at least one embodiment, holes 902 are provided at the second overhang ends 208b, 210b to allow attachment of a mesh to the device, rather than the abdominal wall, which can improve grip offered by the device

### III. Example Method of Pre-Programming Smart Three-Dimensional (3D) Hernia Plug

As explained previously, the smart 3D hernia plug may be manufactured from a shape-memory polymer (SMP) having shape memory capabilities. In accordance with embodiments herein, the SMP may be a thermo-responsive SMP such that the hernia plug transforms from the pre-activation state (FIG. 2B) to the post-activation state (FIG. 2A) in response to a thermal stimuli. In other embodiments, the SMP forming the hernia plug may be selected to respond to any other suitable stimuli.

Reference is now made FIG. 1E, which shows a process flow 100e for a method of pre-programming a thermo-responsive SMP to exhibit shape memory capabilities.

While the process flow 100e is characteristic of a normal process for pre-programming SMPs to have shape memory effect (SME), the process has been described in the context of the unique application of manufacturing a smart 3D hernia plug 200, in accordance with the teachings herein.

As shown, at 160e, the hernia plug 200 is initially manufactured (e.g., printed or fabricated) using SMP material at an ambient temperature below the SMP's glass transition temperature (T_{g}) (e.g., in the region 150c in FIG. 1D).

The shape of the SMP, at 160e, may correspond to the SMP's desired “permanent shape” (or the original shape). For example, at 160e the permanent shape corresponds to the plug's post-activation shape (FIG. 2A). That is, the SMP is printed or molded to shape a hernia plug 200 having the desired post-activation shape.

At 162e, the pre-manufactured SMP is subject to ambient temperatures that exceed the glass transition temperature (T_{g}). This, in turn, causes the SMP to transition into the rubbery state 150d (FIG. 1D). In the rubbery state, the SMP can be flexibly deformed into a desired deformed shape. This deformed shape becomes a new temporary shape once the SMP is exposed to ambient temperatures below the glass transition temperature (T_{g}) at 164e.

For example, in manufacturing the hernia plug 200, once manufactured, the hernia plug 200 may be deformed into its desired pre-activation shape (FIG. 2A) at 162e, and further cooled down to maintain this shape at 164e. In the pre-activated state, the hernia plug may be inserted into a conveyance device (e.g., a laparoscope) (FIG. 3). A pressure force constraint is also applied to the plug to maintain its shape configuration in the temporary shape 164e. For example, the force constraint may be applied by the conveyance device.

At a subsequent point in time, e.g., at deployment at the hernia site—at 166e, once the SMP is again exposed to ambient temperatures above the glass transition temperature (T_{g}), the SMP may transform from its temporary shape (164e) back to its permanent shape (160e)(e.g., also known as shape recovery). For example, at 166e, the hernia plug 200 may transform from its temporary pre-activation state (FIG. 2B), to its post-activation state (FIG. 2A). In other words, thermal stimuli, above glass transition temperature (T_{g}), induces shape recovery and automatically transforms the hernia plug 200 into the permanent post-activation shape.

In view of the foregoing, it will be appreciated that the inventors have applied the process of pre-programming SMPs to a novel application involving a smart 3D hernia plug having a unique structural design.

### IV. Example Method of Manufacturing Smart Three-Dimensional (3D) Hernia Plug

In at least one embodiment, the smart hernia plug 200 is manufactured from a thermo-responsive SMP having a glass transition temperature (Tg) roughly equivalent to the internal human body temperature (e.g., 37° C., or a range of 35° C.-40° C.). In this manner, upon conveyance of the plug 200 to the hernia site, the body temperature may act as a thermal stimuli to transform the SMP to the rubbery or flexible state, corresponding to the post-activation state (FIG. 2A) (e.g., T>Tg)(e.g., 166e in FIG. 1E).

In general, thermo-responsive SMPs are categorized in two main groups: (i) thermosets, and (ii) thermoplastics. It has been appreciated that polyurethane based thermoplastic SMPs have a number of advantages that are relevant to the disclosed hernia plug 200, including ease of processing, ability to display excellent shape memory effect (SME) as well as being light weight. Further, polyurethane-based SMPs have a reasonable range of glass transition temperatures (Tg) and “fast unrolling time”.

In view of the above, and in accordance with at least some embodiments, the hernia plug 200 is manufactured from shape memory polyurethanes (SMPUs), which are thermoplastics that can be heated above a certain temperature to be reprocessed and given different shapes, multiple times (e.g., 162e in FIG. 1E).

Generally, the crystalline regions of SMPUs are hard and brittle in nature, and SMPUs maintain these characteristics at temperatures below the melting temperature. Further, the amorphous regions of SMPUS are soft and only display hard and brittle characteristics below the glass transition temperature (T_{g}) (150c in FIG. 1D). Beyond the T_{g}, but under the melting temperature, the amorphous region transitions from a glassy to rubbery state (150d in FIG. 1D), making it softer and more flexible while the crystalline region remains in the glassy state. This allows the elastic modulus of the amorphous region to reduce, and the SMPU is able to be transform to a temporary shape (162e in FIG. 1E). This shape may be fixed if the temperature is cooled to under the Tg and the appropriate constraints are applied to prevent the temporary shape from changing (164e in FIG. 1E). The reduction of temperature under the Tg results in the amorphous region returning to its glassy state. Heating above the Tg after the temporary shape is programmed, will cause the structure to retain its original shape (166e in FIG. 1E) because of the elastic energy stored in the crystalline region of the SMPU, as the amorphous region softens and is unable to retain the temporary shape. It is important to note that the amorphous regions are responsible for retaining the temporary shape (164e in FIG. 1E) while the crystalline regions are responsible for retaining the permanent or original shape (160e, 166e in FIG. 1E).

In some embodiment, the SMPU, used for manufacturing the hernia plug, is an MM4520 thermoplastic polyurethane shape memory polymer. MM4250 SMPUS's have a glass transition temperature (Tg) of substantially 45° C. However, since the ambient condition of the smart plug is moist (e.g., inside the human body), a drop in T_{g} is expected, which would result in a lower T_{g} than 45° C., and more proximal to the vicinity of internal human body temperature, e.g., approximately 37° C. In at least one embodiment, the hernia plug can be manufactured from an MM4520 manufactured by SMP Technologies®. In some other embodiments, other polyurethane based SMPs may be used, including MM5520, which has a Tg of 55° C.

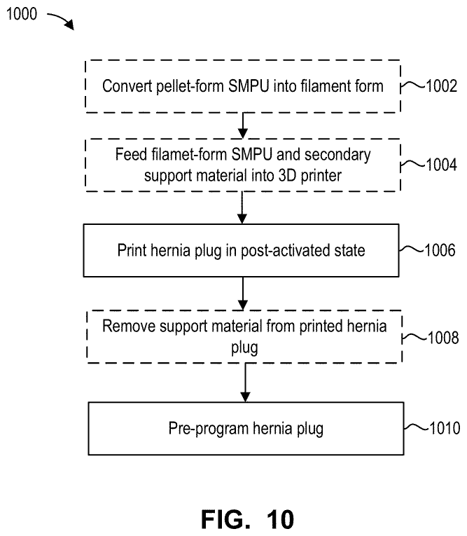

Reference is now made to FIG. 10, which shows a process flow for an example embodiment of a method 1000 for printing a smart 3D hernia plug using SMPU, and using a three-dimensional (3D) printer. The method 1000 can be used to print the hernia plug 200 in its desired permanent, or post-activated shape (160e in FIG. 1E). In various cases, method 1000 may be performed at ambient temperatures that are below the selected SMPU's glass transition temperature (T_{g}).

In particular, method 1000 provides an example process for manufacturing the embodiment of the smart hernia plug 200 exemplified in FIG. 9. Further, while the method 1000 is explained with reference to MM4520 SMPUs, it will be understood that the same method can be applied using other types of SMP materials.

As shown, at 1002, the SMPU may be initially in pellet-form. Accordingly, as an initial step, the SMPU material may be converted into a filament form. This allows feeding the SMPU into a 3D printer, such as a Fused Deposition Modeling (FDM) printer. In other cases, act 1002 may not be necessary if the SMPU is already received in a filament form.

In at least one embodiment, where the SMPU is MM4520 SMPU—at 1002, the pellets are initially dried for a pre-defined number of hours (e.g., 12 hours) in a vacuum oven. For example, the vacuum oven may be a Lindberg/Blue M from Thermo Fisher Scientific Inc.® oven operated at 80° C. This, in turn, may remove and prevent the ingress of moisture in the material, which could potentially form voids in the filament.

The dried pellets are then subject to polymer melt-extrusion. In some embodiments, the polymer melt-extrusion is performed using a 3 mm circular cross section die using a Brabender™ single screw extruder® attached to a drive system to pull and wind the filament. In at least one embodiment, the extruder heating zones are set to 170° C., 180° C., 190° C. and 195° C. for the four heating zones of the extruder, ordered from feeder to the nozzle of the extruder (Table 2). The extrusion rate and pulling speeds may be kept constant, e.g., at 15 rotations per minute (CPM).

TABLE 1
Summary of Extrusion Heating Zone Temperatures for Extruder

| Heating Zone | 1 (Feeder) | 2 | 3 | 4 (Nozzle) |
|---|---|---|---|---|
| Temperature | 170 | 180 | 190 | 195 |
| (° C.) | | | | |

Once the filament is extruded, it is still exposed to the atmospheric humidity and is able to absorb moisture. For this reason, the excess moisture may be removed by drying the filaments in a vacuum oven (e.g., using the Lindberg/Blue M from Thermo Fisher Scientific Inc.®) for a pre-defined period of time (e.g., 12 hours) and sealed in a vacuum bag, to prevent moisture ingress, until printing is required.

At 1004, the filament-form SMPU, as well as a secondary support material, are fed (e.g., supplied) into the 3D printer.

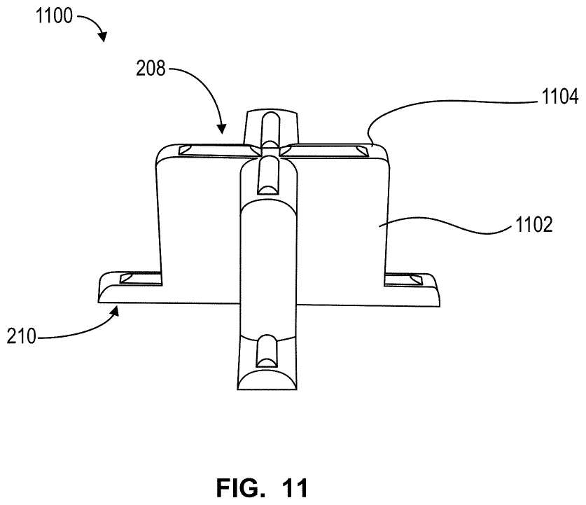

To better appreciate the function of the secondary support material, reference is briefly made to FIG. 11. As shown, during printing, the secondary support material 1102 acts as a scaffold, or support frame, for the SMPU material 1104 as it is being printed in the shape of the hernia plug, e.g., the post-activated shape. That is, the secondary material 1102 stabilizes the structure of the print sample, as well ensures the quality of the print sample. After the printing is complete, the secondary support material may be removed (e.g., dissolved or mechanically removed), such that only the SMPU material 1104 remains (e.g., as shown in FIG. 9). In some embodiments, the secondary material may comprise water-soluble support material, such that the secondary support material 1102 immediately dissolves upon application of water to the print sample.

To this end, to facilitate manufacturing of the support structure 1102, a dual nozzle 3D printer is used to manufacture the hernia plug 200. In particular, one nozzle is used to print the SMPU material 1104, and the other nozzle is used to print the support material 1102. In at least one embodiment, a dual nozzle Ultimaker 3® (UM3) Fused Deposition Modeling (FDM) printer is used.

In at least one embodiment, the secondary support material 1102 is selected as polyvinyl alcohol (PVA). PVA is supported by the Ultimaker® 3 (UM 3) FDM printer, with benefits such as non-toxicity, reliable adhesion to a number of commonly used filaments for 3D printing, and is complete biodegradability with good thermal stability and complete water solubility.

In other embodiments, secondary support material 1102 may not be used to fabricate the hernia plug. Accordingly, at act 1004, only the SMPU material is fed into the 3D printer. If this is the case, a single nozzle FDM printer may be sufficient, such as the Ultimaker®2 FDM printer.

In some embodiments, act 1004 may not be necessary if the printing material (e.g., the filament-form SMPU and the secondary support material) are already pre-loaded into the 3D printer.

Referring still to method 1000, at 1006, the hernia plug is printed, using the 3D printer, in the post-activated shape. For example, the 3D printer can be programmed to print the desired post-activation shape using the SMPU material, and in some cases, the secondary support material.

Table 2 summarizes example optimized printing parameters selected for printing the hernia plug design exemplified in FIG. 9 using a UM3® dual-nozzle FDM printer. In this example, the SMPU material was selected as MM4520 SMPU, and the support material was selected to be PVA. The operating parameters in Table 2 are based off repeated efforts to optimize printing parameters against surface quality, print integrity and overall printing success as per design benchmarks set out in the fabrication phase.

TABLE 2
Summary of Operating Parameters

| Printing Parameters | Value | Unit |
|---|---|---|
| Layer Height | 0.2 | Mm |
| Nozzle Speed | 10 | mm/sec |
| Nozzle Temperature (MM4520) | 220 | ° C. |
| Nozzle Temperature (PVA) | 220 | ° C. |
| Print Bed temperature (PVA) | 70 | ° C. |
| Infill | 100 | % |
| Support Layer Count | 3 | — |
| Primary Nozzle Size | 0.4 (BB) | mm |
| Secondary Nozzle Size | 0.4 (BB) | mm |

Printing an individual sample with supports required approximately four hours to print, and in an effort to increase efficiency and to reduce potential variations between printed samples—a batch production approach was considered where a set of five samples were produced at the same time. All of the samples in the batch, were printed simultaneously, since the UM3 did not support the fabrication of one sample at a time with the use of dual nozzle. The batch printing took approximately 26 hours to complete and generally resulted in lower number of failures as compared to the single print approach, therefore allowing for more available samples for the characterization phase of this study.

At 1008, if the hernia plug 200 is printed with secondary support material, then the secondary support material may be removed. For example, this can involve dissolving the secondary support material (e.g., by applying water to the printed hernia plug).

At 1010, once the hernia plug is manufactured in the post-activation state, the plug can be pre-programmed as previously described with reference to FIG. 1E to define the plug's pre-activation shape (e.g., acts 162e and 164e).

### V. Evaluating Mechanical Performance of Example Smart Three-Dimensional (3D) Hernia Plug

The following section evaluates the mechanical performance of a hernia plug 200 have a designed as exemplified in FIG. 9. As noted earlier, the mechanical performance evaluates how the hernia plug design performs in respect of the desired mechanical properties of a hernia plug device.

As inguinal hernia impairs the mechanical function of the abdominal wall, it assists to understand the mechanical properties of the healthy abdominal wall. Based on the law of Laplace and maximum inter-abdominal pressure (IAP), a mesh with a tensile strength of 16 N/cm was previously determined be sufficient for the use of hernia repair (C. N. Brown and J. G. Finch, “Which mesh for hernia repair?,” Annals of the Royal College of Surgeons of England, vol. 92, no. 4. Royal College of Surgeons of England, pp. 272-278, May 2010, doi: 10.1308/003588410X12664192076296, herein after “C. N. Brown and J. G. Finch”). While this is believed to be the physiological strength of the human abdominal wall, most commercial meshes are capable of withstanding significantly higher tensile forces. Since the organs inside the abdominal cavity are held in place by the abdominal wall, it experiences a constant state of tension. Any meshes that are attached to it (FIG. 1B), also experience similar tensile forces, and for the case of a hernia, must be able to withstand these tensile force

The intra-abdominal pressure is reported in literature to be approximately 170 mmHg, resulting in meshes being used to treat hernia repairs required to withstand at least 180 mmHg which is equivalent to 32 N/cm (C. N. Brown and J. G. Finch). Owing to the difference in application of the proposed smart plug, the force loading is different from conventional meshes. Considering the weight of the contents of the abdominal cavity, the smart 3D hernia plug 200 is believed to be in a constant state of compression and is accordingly designed to withstand at least 180 mmHg or approximately 24 KPa.

On this note, the mechanical performance of the disclosed hernia plug was considered to determine the endurance and the integrity of the plug using: (a) a single compression test, and (b) a cyclical compression test.

As provided herein, both tests were conducted on a 3D hernia plug 200 as exemplified in FIG. 9, and using a Bose® ElectroForce® 3200 Series 3 test instrument equipped with a 450 N load cell inside a temperature chamber. The temperature chamber allows setting and maintaining a certain temperature while conducting tests, which is important since all the characterization tests conducted on the smart plug is at the normal resting human body temperature of 37° C.

Once the samples were printed in a batch, the samples were soaked in tap water for a week, which dissolves the secondary supports as well as allow the intake of water to simulate the moist internal environment of the human body. Upon soaking, the respective tests were conducted in the Bose® machine and data was recorded and analyzed.

### (i) Example Compression Test

The first test to characterize the mechanical properties of the 3D hernia plug was performed by conducting a single compression test to determine the stiffness and maximum force of the 3D hernia plug before compaction.

The single compression test was conducted by keeping the bottom plunger of the Bose® machine stationary, which was attached to the 450 N load cell of the Bose® machine with a resolution of 10%, and moving the top plunger at a speed of 1 mm/sec by a total displacement of 12 mm followed by complete retraction. The temperature was kept constant at 37° C., and the force exerted by the sample was recorded with respect to displacement.

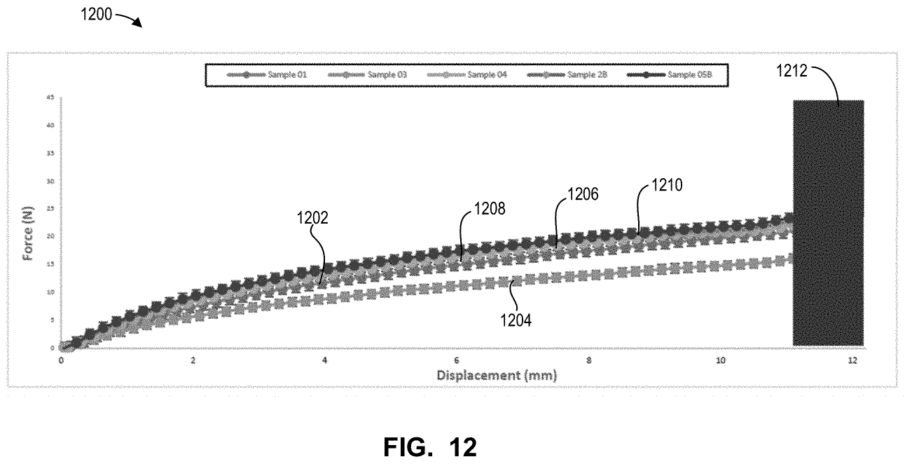

FIG. 12 shows a plot 1200 of the results of the compression test applied to five different 3D hernia plug samples 1202, 1204, 1206, 1208, 1210, all designed as exemplified in FIG. 9. Plot 1200 illustrates force applied (Newtons (N)) versus displacement (millimeter (mm)) for each of the test samples.

As shown, all samples yielded similar graphs. The two important parameters generated from the compression test were: (i) the stiffness of the 3D hernia plug, as well as (ii) the maximum force before compaction. The 3D hernia plug 200 experienced full compaction at approximately just over 11 mm of compressive displacement. The full compaction region is represented by region 1212 in plot 1200. As the samples experienced full compaction, the force experienced by the plug increased exponentially with smaller increments of displacement. The significance of illustrating the compaction region 1212 is to illustrate that despite compaction, the sample did not show signs of failure. Since the sample exhibited compaction at 11 mm, the value of maximum force therefore is taken at 11 mm of compressive displacement for all samples. The value of all five samples were recorded and averaged to generate a single value, for reporting purposes.

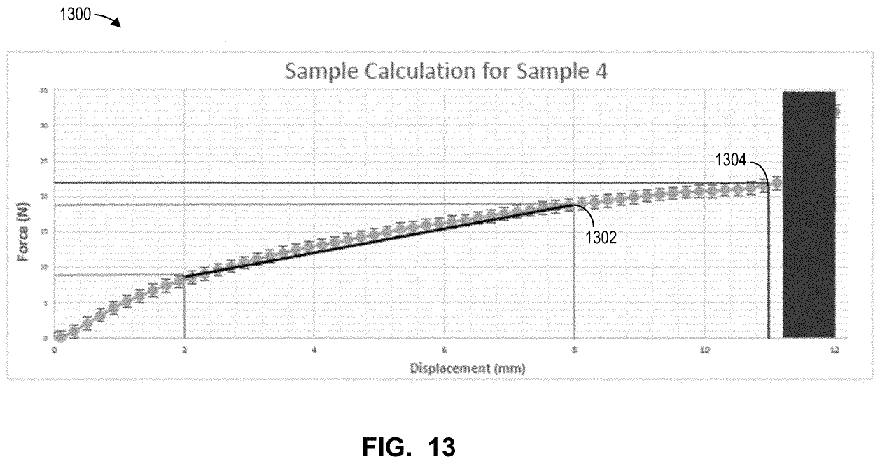

Turning to the stiffness, the stiffness of the 3D hernia plug was determined by calculating the slope of individual plots, as shown in plot 1300 of FIG. 13. In particular, FIG. 13 shows a plot 1300 of the data results consequent of applying the compression test to sample 1206, from plot 1200.

As shown, the maximum compressive force experienced by the sample is 22 N. The slope to calculate stiffness is measured from 2 mm to 8mm (e.g., identified by points 1302 and 1304, respectively), and is calculated to be 1.667 N/mm which is equivalent to 16.67 N/cm as shown by Equation (1), below:

$$Stiffness = Slope of Graph = \frac{{y}_{2} - {y}_{1}}{{x}_{2} - {x}_{1}} = \frac{19 - 9}{8 - 2} = \frac{10}{6} = 1.667 \frac{N}{mm} \qquad ( 1 )$$

Similar to this model calculation, the remaining samples were examined and the values for maximum force and stiffness were calculated, tabulated, as shown in Table 3.

TABLE 3
Summary of Results for Single Compression Test

| Sample No. | Maximum Force (N/cm) | Stiffness (N/mm) |
|---|---|---|
| 1 | 19.1 | 1.75 |
| 2 | 20.0 | 1.60 |
| 3 | 14.5 | 1.25 |
| 4 | 20.0 | 1.67 |
| 5 | 20.1 | 1.61 |
| Average | 18.74 | 1.58 |

Based on these value, it was determined that the average maximum force was measured to be 18.74 N/cm, and that the disclosed 3D hernia plug is within acceptable range of 11.1 N/cm to 100.9 N/cm displayed by alternative meshes. It has also been appreciated that alternative meshes display a wide range of values of maximum force and stiffness, which is attributed to the type of mesh being either a heavy weight mesh or light weight mesh as well as difference due to material properties used to manufacture the mesh.

It is noted that, upon full compaction, the 3D hernia plug 200 did not appear to show any signs of failure and recovered to its post-activation shape without any major signs of plastic deformation. The value calculated is therefore considered to be conservative value, and while although it is not a direct comparison to these meshes, it is able to endure similar loading conditions with ease. Further, the stiffness of the disclosed hernia plug was 1.58 N/mm, which is well within the acceptable range.

The compression test was therefore successful in validating results and demonstrated that the disclosed smart plug 200 in FIG. 9 was, at minimum, at par with alternatives in terms of the mechanical parameters discussed.

### (ii) Example Cyclical Compression Test

The second test conducted to characterize the mechanical properties of the disclosed smart hernia plug involved performing a cyclic compression test to study the effects of repeated and cyclic loading on the smart plug.

The cyclic compression test was conducted under similar test conditions with the only change being the displacement of 11 mm and instead of a single test being conducted, a total of 10 continuous cycles were performed, respective force and displacement values recorded and then analyzed, as similarly done in S. Roman, N. Mangir, L. Hympanova, C. R. Chapple, J. Deprest, and S. MacNeil, “Use of a simple in vitro fatigue test to assess materials used in the surgical treatment of stress urinary incontinence and pelvic organ prolapse,” Neurourol. Urodyn., vol. 38, no. 1, pp. 107-115, Jan. 2019, doi: 10.1002/nau.23823 (also referred to herein as Roman, et al). This process was repeated for three samples.

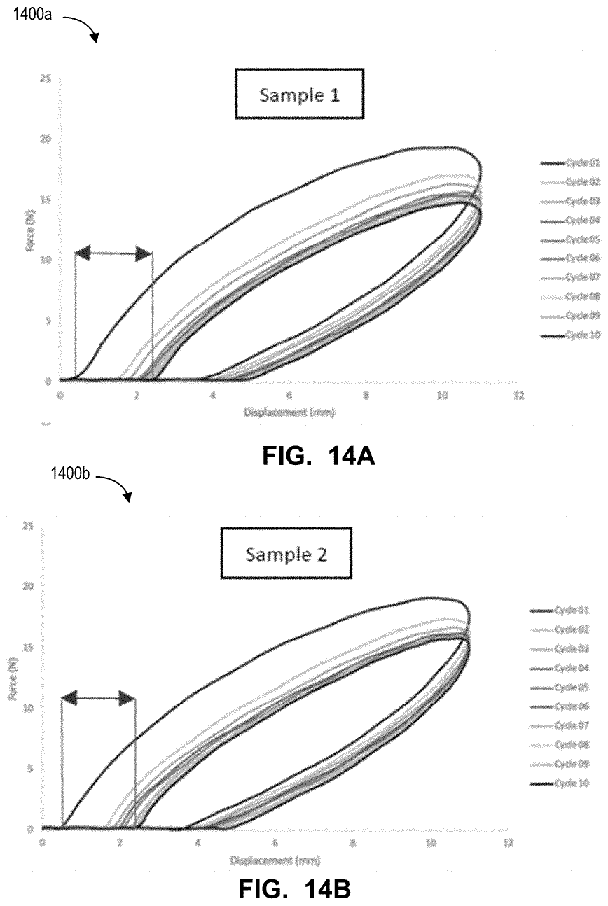

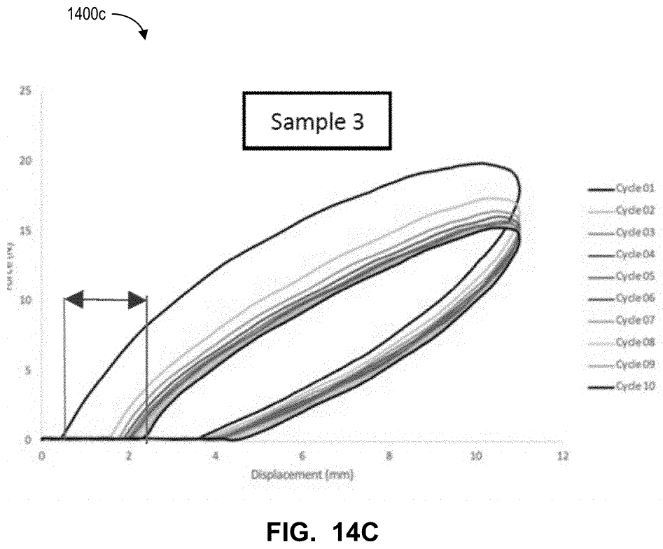

FIGS. 14A-14C illustrate plots 1400a-1400c, respectively, which show the results of the cyclical compression test for three different sample.

All samples experienced approximately 2 mm of plastic deformation under cyclic loading, with the plastic deformation between the first and tenth cycle of each sample marked in plots 1200a-1200c. The average plastic deformation for all three samples was calculated to be 7.40% of compressive strain and was a calculated using Equation (2), below:

$$\frac{( Displacement of 10 th cycle - Displacement of 1 st Cycle )}{Total height of sample} = \frac{2 mm}{27 mm} \% = 7.4 \% \qquad ( 2 )$$

Similar to the work of Roman et al., which conducted this test to study the effects of cyclic tensile loading on a few meshes, a similar test was conducted under cyclic compressive loading. While the work of Roman et al. was under tension and similar results were replicated under compression, it is important to note this was done due to the difference in loading conditions that experienced by the smart plug and conventional meshes. Roman et al. studied commercial meshes which displayed percentage deformation ranging from 0.58% to 8.51%. This value was calculated by measuring the deformation between the first cycle and last cycle of deformation during cyclic loading. The disclosed embodiments of the 3D hernia plug displayed approximately 7.40% of permanent deformation, which is within range of those available in the study by Roman et al. It also displays a similar cyclic loading pattern and may be deemed acceptable keeping in mind that this is not a direct comparison and small differences are expected.

### (iv) Example Shape Memory Characteristics

The shape memory characteristics mainly involved the study of the shape recovery ratio and the shape recovery speed. This was performed to determine the deployment speed and activation properties. The shape memory characteristics were measured and analyzed separately for each of the short overhangs 208, the waist portion 206 and the long overhangs 210.

To evaluate the shape memory characteristics of the smart hernia plug, the hernia plug sample, in accordance with embodiment exemplified in FIG. 9, was initially intended to be preheated to 50° C. (T_{g}+5° C.) to program the pre-activation shape. However, due to soaking of the sample in water for a week to dissolve the supports and replicate the abdominal environment, the sample was able to be programmed at a much lower temperature while displaying significant flexibility even at room temperature. This phenomenon was attributed to the lowering of T_{g} due to the absorption of water.

In light of this, the oven (Lindberg/Blue M from Thermo Fisher Scientific Inc.®) was preheated to 45° C., and the sample was placed in the oven for a duration on 15 minutes and an initial image was taken as reference for that particular sample. To program the pre-activation shape onto the sample, the sample was pushed into a brass tube of internal diameter of 12.5 mm and placed inside an ice bath. The oven was allowed to cool and was then reheated to 37° C. while the enclosed sample soaked in the ice bath.

The brass tube was removed underwater inside the ice bath to ensure that the sample does not activate and then immediately placed inside the oven. The activation of the sample was recorded using a Basler acA3800-10gm GigE® camera The camera was calibrated to take pictures of the deploying smart plug at a frequency of 4 Hz. A regular paper tape was also kept in the shot and was pre-measured and placed such that the center of the tape would overlap with the suspended smart plug in an effort to negate any errors due to depth of field. An image sample of the pre-activation and post-activation capture by the camera.

These series of images were collected and then further processed in ImageJ® to measure the angle of deployment of the short and long overhangs as well as the expansion of waist. To measure this information, a few points were marked as reference and were tracked and measured manually with the appropriate scale which was calibrated using a paper tape in the bottom of the image. This information was then analyzed to measure the shape recovery ratio which is defined as the ratio of shape recovery for each component from pre-activation to post-activation and finally compared to the original reference taken in the start.

The recovery speed was also calculated using the above technique and is defined as the recovery of shape with respect to time while also providing us with total time required to activate completely and attain post activation shape

To this end, the shape memory characteristics were determined by investing two parameters namely, (i) shape recovery ratio, and (ii) shape recovery speed. Once the pictures of the activating sample were taken and data analyzed , a “recovery ratio vs time” graph was plotted to determine these two parameters. The recovery ratio was a normalized number determined from the ratio of the change in displacement or angle with respect to the original reference picture taken before shape memory programming. The slope of this graph was considered as the shape recovery speed while the recovery ratio value at 240 seconds (e.g., four minutes) after activation was considered as final shape recovery ratio.

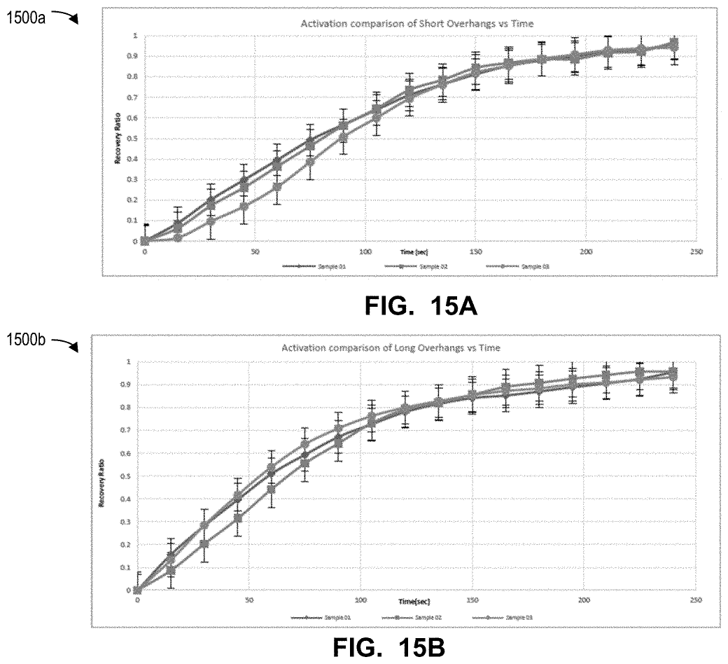

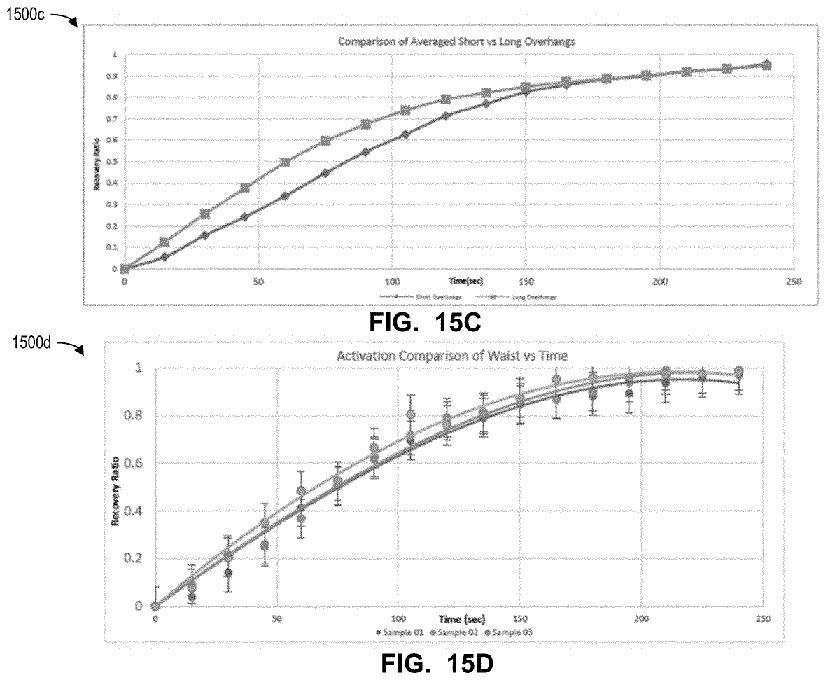

The raw data obtained for the short and long overhangs was further smoothened using a five-point moving average using MATLAB® and plotted accordingly as shown in plots 1500a-1500d in FIGS. 15A-15D, respectively.

As shown in plots 1500a of FIG. 15A and plot 1500b of FIG. 15B, with acceptable variation among samples, the deployment of the short and long overhangs experienced shape recovery of more than 90% within 240 seconds of activation (e.g., four minutes).

As shown in plot 1500c of FIG. 15C, the deployment of the long overhangs is faster in comparison to the short overhangs, with the averaged speed of activation being calculated by measuring the slope of the two graphs from 0 to 100 seconds. The averaged activation speed for the short and long overhangs is calculated to be 0.0060 sec-1 and 0.0073 sec-1 respectively which is equivalent to 0.540°/sec and 0.657°/sec, respectively.

The raw data obtained for the waist of the smart plug was mapped using curve fitting and the data points subsequently plotted as polynomial of the third degree. With acceptable variation between samples, the activation comparison of the waist can be seen in plot 1500d of FIG. 15D. Similar to the short and long overhangs, the waist also exhibits shape recovery of greater than 90% by 240 seconds. The samples also demonstrated an average waist activation speed of 0.0067 sec-1 which is equivalent to 0.0181 mm/sec.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative of the invention and non-limiting and it will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

1. A three-dimensional (3D) hernia plug, comprising:
a waist portion extending, along a longitudinal extension axis, between a first waist end and a second waist end;
one or more first overhangs coupled to the first waist end; and
one or more second overhangs coupled to the second waist end,
wherein the hernia plug is configured to transform between a pre-activation state and a post-activation state.

2. The hernia plug of claim 1, wherein the hernia plug is configured to change shape between the pre-activation state and a post-activation state.

3. The hernia plug of claim 1, wherein in the pre-activation state, each of the one or more and second first overhangs extends substantially parallel to the extension axis, and in the post-activation state, each of the one or more first and second overhangs extends substantially orthogonal to the extension axis.

4. The hernia plug of claim 2, wherein the hernia plug translates from the pre-activation state to the post-activation state in response to a thermal stimuli.

5. The hernia plug of claim 4, wherein the thermal stimuli is an ambient temperature in a range of 35° C. to 40° C.

6. The hernia plug of claim 4, wherein the hernia plug is manufactured from a smart memory material.

7. The hernia plug of claim 6, wherein the thermal stimuli corresponds to the glass transition temperature (T_{g}) of the smart memory material.

8. The hernia plug of claim 7, wherein the smart material polymer comprises one or more of a shape memory polyurethane (SMPU) and shape memory allot (SMA).

9. The hernia plug of claim 1, wherein the hernia plug is deployable at a hernia-defect site in the post-activated state, the hernia-defect site comprising an inguinal hernia-defect side, and the one or more first overhangs are configured to engage a groin-side of an abdominal wall, and the one or more second overhangs are configured to engage an abdomen-side of the abdominal wall, such that the hernia plug is self-gripping.

10. The hernia plug of claim 1, wherein each of the one or more first and second overhangs comprises one of a planar member and a rod-like elongate member, and the waist portion comprises one of, (i) one or more connected continuous members, and (ii) one or more spaced discrete members.

11. The hernia plug of claim 1, wherein the waist portion expands in an axis orthogonal to the extension axis in the post-activated state.

12. A method of manufacturing a three-dimensional (3D) hernia plug, comprising:
fabricating, using a shape memory material, the hernia plug in a post-activation shape, wherein the printing occurs in an ambient temperature that is below a glass transition temperature (T_{g}) of the smart polymer material;
deforming the hernia plug into a pre-activation shape, wherein the deforming occurs while the ambient temperature is above the glass transition temperature (T_{g}); and
reducing the ambient temperature is to below the glass transition temperature (T_{g}) to allow the pre-activation shape to correspond to a temporary shape of the hernia plug.

13. The method of claim 12, wherein the shape memory material comprises one of a shape-memory polyurethane (SMPU).

14. The method of claim 13, wherein the smart material polymer comprises an MM4520 SMPU.

15. The method of claim 12, wherein in the post-activation shape, the hernia plug is printed to comprise:
a waist portion extending, along a longitudinal extension axis, between a first waist end and a second waist end;
one or more first overhangs coupled to the first waist end; and
one or more second overhangs coupled to the second waist end, and
each of the one or more first and second overhangs extends substantially orthogonal to the extension axis.

16. The method of claim 15, wherein in the pre-activation state, each of the one or more and second first overhangs extends substantially parallel to the extension axis.

17. The method of claim 15, wherein the fabrication further comprises fabricating the hernia plug using a secondary support material.

18. The method of claim 17, wherein after the fabrication and before the deforming, the method further comprises removing the secondary support material.

19. The method of claim 18, wherein the secondary support material comprises water-soluble material, and removing the secondary support material comprises exposing the fabricated hernia plug to water.

20. The method of claim 15, wherein the fabrication is performed using a three-dimensional (3D) printer.
